(19) United States
(12) Patent Application Publication
(10) Pub. No.: US 2026/0096757 A1
(43) Pub. Date: Apr. 9, 2026

(54) IMPLANTABLE MENTAL STATE MONITOR

(71) Applicant: Medtronic, Inc., Minneapolis, MN (US)

(72) Inventors: Richard J. O'Brien, Hugo, MN (US); Todd M. Zielinski, McGrath, MN (US); Randal C. Schulhauser, Phoenix, AZ (US); Ekaterina B. Morgounova, Minneapolis, MN (US); Bruce D. Gunderson, Plymouth, MN (US); Xusheng Zhang, Shoreview, MN (US); Paul G. Krause, Mahtomedi, MN (US); Catherine R. Condie, Shoreview, MN (US); Anna J. Malin, Paradise Valley, AZ (US); Taycia L. Brandon, Otsego, MN (US)

(21) Appl. No.: 19/112,758

(22) PCT Filed: Oct. 6, 2023
(86) PCT No.: PCT/US2023/034603

§ 371 Date: Mar. 18, 2025

Related U.S. Application Data
(60) Provisional application No. 63/378,814, filed on Oct. 7, 2022.
(60) Provisional application No. 63/386,120, filed on Dec. 5, 2022.

(51) Int. Cl. A61B 5/16; A61B 5/00; A61B 5/0205; A61B 5/287; A61B 5/293

(52) U.S. Cl. CPC A61B 5/165; A61B 5/0022; A61B 5/02055; A61B 5/287; A61B 5/293; A61B 5/4803; A61B 5/4815; A61B 5/7264; A61B 2562/0219

(57) ABSTRACT

A system comprises one or more implantable monitoring devices configured to continuously sense a plurality of physiological signals of a subject and collect parameter data of the subject based on the sensed physiological signals. At least one monitoring device of the one or more monitoring devices comprises a housing configured for subcutaneous implantation in the subject and a plurality of electrodes positioned on the housing. The at least one monitoring device is configured to continuously sense at least one physiological signal of the plurality of physiological signals via the plurality of electrodes. The system further comprises processing circuitry configured to determine a mental state of the subject based on at least one of the sensed physiological signals or the parameter data.

This application claims the benefit of priority from U.S. Provisional Ser. No. 63/386,120 , filed Dec. 5, 2022, and U.S. Provisional Ser. No. 63/378,814, filed Oct. 7, 2022, the entire contents of each of which are incorporated herein by reference.

### FIELD

This disclosure generally relates to systems including medical devices and, more particularly, to monitoring of patient health using such systems.

### BACKGROUND

A variety of devices are configured to monitor physiological signals of a patient.

Such devices include implantable or wearable medical devices, as well as a variety of wearable health or fitness tracking devices. The physiological signals sensed by such devices include as examples, electrocardiogram (ECG) signals, respiration signals, perfusion signals, activity and/or posture signals, pressure signals, blood oxygen saturation signals, body composition, and blood glucose or other blood constituent signals. In general, using these signals, such devices facilitate monitoring and evaluating patient health over a number of months or years, outside of a clinic setting.

Mood disorders can impact individuals' ability to function at work and at home and can lead to a cycle of self-destructive behaviors and damaged relationships. Example mood disorders include depression, anxiety, bipolar disorder, substance-abuse mood disorders, anorexia, post-partum mood disorders, post-traumatic stress disorder (PTSD), and menstrual-related mood disorders, such as pre-menstrual syndrome, pre-menstrual dysphoric disorder, and perimenopausal depression.

Stress, trauma, worsening health, or other difficult life events can exacerbate mood disorder symptoms. Life changing medical events that require rehabilitation, such as a coronary artery bypass graft (CABG) procedure, a stroke, a heart attack, or a traumatic injury, frequently generate symptoms of depression that can complicate the success of the rehabilitation. Chronic illnesses, such as cancer and heart failure or other heart disease, may lead to symptoms of mood disorders, and mood disorders may negatively impact the course of chronic illness. Stressful life events such as the death of a spouse, child or a close family member, divorce and separation, job loss, residential relocation or homelessness, imprisonment, and even normal life transitions such as marriage, retirement, pregnancy, or the arrival of a child, can trigger or exacerbate mental health issues. Depression can increase the risk of development of coronary artery disease and adverse cardiac events such as heart attack or blood clots, as well as asthma, autoimmune diseases, respiratory infections, and mortality. Receipt of defibrillation therapy may be traumatic and can also result in symptoms of depression or anxiety. Women are twice as likely to develop depression as men, and menstruation can exacerbate mood disorder symptoms.

### SUMMARY

In general, the disclosure describes techniques for continuously monitoring the mental state of a subject using one or more implantable monitoring devices. The one or more implantable monitoring devices are configured to continuously sense a plurality of physiological signals of a subject and collect parameter data of the subject based on the sensed physiological signals. Processing circuitry of the implantable monitoring device(s) or other computing devices/systems may be configured to determine a mental state of the subject based on at least one of the sensed physiological signals or the parameter data.

At least one monitoring device comprises a housing configured for subcutaneous implantation in the subject and a plurality of electrodes positioned on the housing and is configured to continuously sense at least one physiological signal, e.g., an electrocardiogram (ECG) or electroencephalogram (EEG), via the plurality of electrodes. Other physiological signals that may be monitored for the determination of subject mental state include heart rate, heart rate variability, blood pressure, activity, posture, oxygen saturation, skin conductance, tissue impedance, respiration, cough detection, or temperature. In some examples, the processing circuitry may additionally or alternatively determine mental state based on subject movement or subject interaction with smartphones or other computing devices, which may include social media use or contact with friends. In some examples, the processing circuitry may additionally or alternatively determine mental state based on changes in the voice of a subject relative to a baseline. A subcutaneously implantable monitoring device may include a sensor to detect the subject's voice without intervention by the subject or another user. Mental states monitored according to the techniques of this disclosure include states of mood disorders or alertness/fatigue.

The techniques of this disclosure may be implemented by one or more implantable monitoring devices that can continuously (e.g., on a periodic or triggered basis without human intervention) sense the physiological signals while subcutaneously implanted in a patient over months or years and perform numerous operations per second on the physiological signals to enable the systems herein to determine mental states of a subject. Determining mental states of subjects using physiological signals continuously sensed by implantable monitoring devices may provide one or more technical and clinical advantages. For example, using techniques of this disclosure with an implantable monitoring device may be advantageous when a physician or other interested party cannot be continuously present with the subject over weeks or months to evaluate the subject. By detecting changes in mental state based on physiological signals, the techniques of this disclosure may advantageously overcome the above-discussed problems with self-reporting of symptoms of worsening mental states. Using the techniques of this disclosure with an implantable monitoring device may advantageously allow continuous monitoring of subject mental states without requiring subject compliance with self-reporting or wearable monitors. The ability and/or willingness of a subject to comply is negatively impacted by worsening mental states. Additionally, the techniques described herein may be advantageous where performing the operations on the physiological signals described herein on weeks or months of data could not practically be performed in the mind of a physician.

In some examples, the systems of this disclosure may use a machine learning model to more accurately determine the mental state of a subject based on physiological signals and parameter data collected by one or more implantable monitoring devices. In some examples, the machine learning model is trained with a set of training instances, where one or more of the training instances comprise data that indicate relationships between various signals, data, and/or features/parameters derived therefrom, and classifications or other outputs representing possible mental states. Because the machine learning model is trained with potentially thousands or millions of training instances, the machine learning model may reduce the amount of error in determining mental states compared to other techniques for determining the mental state of a subject.

In one example, a system comprises one or more implantable monitoring devices configured to continuously sense a plurality of physiological signals of a subject and collect parameter data of the subject based on the sensed physiological signals, wherein at least one implantable monitoring device of the one or more implantable monitoring devices comprises a housing configured for subcutaneous implantation in the subject and a plurality of electrodes positioned on the housing, wherein the at least one implantable monitoring device is configured to continuously sense at least one physiological signal of the plurality of physiological signals via the plurality of electrodes. The system further comprises processing circuitry of one or more of: the at least one implantable monitoring device; one or more computing devices configured to wirelessly communicate with the one or more implantable monitoring devices; or a cloud computing system configured to communicate with at least one of the one or more implantable monitoring devices or the one or more computing devices. The processing circuitry is configured to determine a mental state of the subject based on at least one of the sensed physiological signals or the parameter data.

In another example, a medical system comprises an insertable cardiac monitor comprising a housing configured for subcutaneous implantation in a subject, the housing having a length between 40 millimeters (mm) and 60 mm between a first end and a second end, a width less than the length, and a depth less than the width, a first electrode at or proximate to the first end, a second electrode at or proximate to the second end, sensing circuitry within the housing, the sensing circuitry configured to continuously sense a plurality of physiological signals including an at least an electrocardiogram of the subject via the first electrode and the second electrode, a memory within the housing, and first processing circuitry within the housing, the first processing circuitry configured to collect parameter data of the subject based on the sensed physiological signals. The system further comprises one or more computing devices in communication with the insertable cardiac monitor, the one or more computing devices comprising second processing circuitry configured to determine a mental state of the subject based on at least one of the sensed physiological signals or the parameter data.

Another example is a method for operating a system comprising one or more implantable monitoring devices to determine a mental state of a subject, wherein at least one implantable monitoring device of the one or more implantable monitoring devices comprises a housing configured for subcutaneous implantation in the subject and a plurality of electrodes positioned on the housing, wherein the at least one implantable monitoring device is configured to continuously sense at least one physiological signal of the plurality of physiological signals via the plurality of electrodes. The method comprises continuously sensing, by the one or more implantable monitoring devices, a plurality of physiological signals of the subject, collecting, by the one or more implantable monitoring devices, parameter data of the subject based on the sensed physiological signals, and determining, by processing circuitry, a mental state of the subject based on at least one of the sensed physiological signals or the parameter data. The processing circuitry comprises processing circuitry of one or more of: the at least one implantable monitoring device; one or more computing devices configured to wirelessly communicate with the one or more implantable monitoring devices; or a cloud computing system configured to communicate with at least one of the one or more implantable monitoring devices or the one or more computing devices.

In another example, a non-transitory computer-readable storage medium comprises program instructions that, when executed by processing circuitry of a medical system, cause the processing circuitry to continuously sense, via one or more implantable monitoring devices, a plurality of physiological signals of the subject, cause the one or more implantable monitoring devices to collect parameter data of the subject based on the sensed physiological signals, and determine a mental state of the subject based on at least one of the sensed physiological signals or the parameter data.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example system configured to determine the mental state of a subject in accordance with one or more techniques of this disclosure.

FIG. 2 is a block diagram illustrating an example configuration of an implantable monitoring device that operates in accordance with one or more techniques of the present disclosure.

FIG. 3 is a block diagram illustrating an example configuration of a computing device that operates in accordance with one or more techniques of the present disclosure.

FIG. 4 is a block diagram illustrating an example configuration of a health monitoring system that operates in accordance with one or more techniques of the present disclosure.

FIGS. 5A-5G are conceptual diagrams illustrating example implantable monitoring devices.

FIG. 6 is a conceptual diagram illustrating an example machine learning model configured to determine a mental state of a subject.

FIG. 7 is a block diagram illustrating training a machine learning model in accordance with one or more techniques of the present disclosure.

FIG. 8 is a flow diagram illustrating an example operation for determining a mental state of a subject according to the techniques of this disclosure.

FIG. 9 is a flow diagram illustrating an example operation for determining a heart failure state of a subject according to the techniques of this disclosure.

FIG. 10 is a flow diagram illustrating an example operation for identifying sound signal segments suitable for voice characteristic measurement according to the techniques of this disclosure.

FIG. 11 is a flow diagram illustrating an example operation for determining subject mental state based on changes in subject voice characteristics according to the techniques of this disclosure.

Like reference characters refer to like elements throughout the figures and description.

### DETAILED DESCRIPTION

A variety of types of implantable and external devices are configured to detect arrhythmia episodes and other acute health events based on sensed ECGs and, in some cases, other physiological signals. External devices that may be used to non-invasively sense and monitor ECGs and other physiological signals include wearable devices with electrodes configured to contact the skin of the patient, such as patches, watches, rings, necklaces, hearing aids, a wearable cardiac monitor or automated external defibrillator (AED), clothing, car seats, or bed linens. Such external devices may facilitate relatively longer-term monitoring of patient health during normal daily activities.

Implantable medical devices (IMDs) also sense and monitor ECGs and other physiological signals and detect acute health events such as episodes of arrhythmia, cardiac arrest, myocardial infarction, stroke, and seizure. Example IMDs include pacemakers and implantable cardioverter-defibrillators, which may be coupled to intravascular or extravascular leads, as well as pacemakers with housings configured for implantation within the heart, which may be leadless. Some IMDs do not provide therapy, such as implantable patient monitors. One example of such an IMD is the Reveal LINQ™ or LINQ II™ Insertable Cardiac Monitor (ICM), available from Medtronic plc, which may be inserted subcutaneously. Such IMDs may facilitate relatively longer-term continuous monitoring of patients during normal daily activities, and may periodically or on demand transmit collected data, e.g., episode data for detected arrhythmia episodes, to a remote patient monitoring system, such as the Medtronic CareLink™ Network.

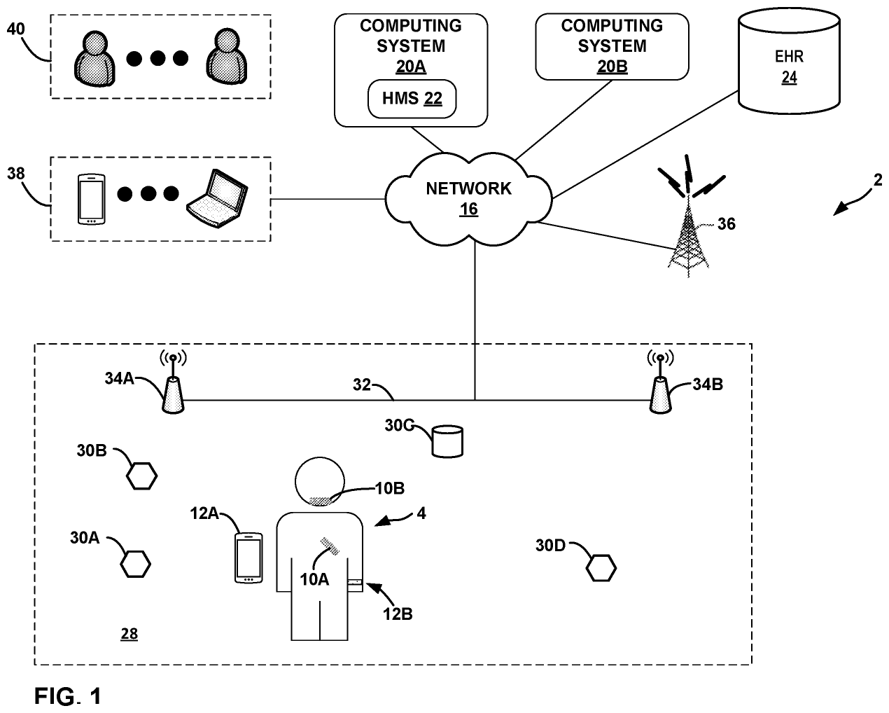

FIG. 1 is a block diagram illustrating an example system 2 configured to monitor a mental state of a patient 4, which is an example of a subject, and to responsively communicate with one or more users, in accordance with one or more techniques of this disclosure. The example techniques may be used with one or more patient sensing devices, e.g., one or more of IMDs 10A and 10B (collectively “IMDs 10”), which may be in wireless communication with one or more patient computing devices, e.g., patient computing devices 12A and 12B (collectively, “patient computing devices 12”). Although not illustrated in FIG. 1, IMDs 10 include electrodes and/or other sensors to sense physiological signals of patient 4 and may collect and store sensed parameter data based on the signals. One or more elements of system 2 may determine a mental state of patient 4 based on the collected data. IMDs 10 are examples of implantable monitoring devices.

IMD 10A may be implanted outside of a thoracic cavity of patient 4 (e.g., subcutaneously in the pectoral location illustrated in FIG. 1). IMD 10A may be positioned near the sternum near or just below the level of the heart of patient 4, e.g., at least partially within the cardiac silhouette. In some examples, IMD 10A takes the form of the Reveal LINQ™ or LINQ II™ ICM. IMD 10B may be a cranial sensor device implanted subcutaneously on the back or side of the head or neck, e.g., as described in commonly assigned U.S. Patent Publication No. 2021/0251497, titled “System and Method for Detecting Strokes,” and commonly assigned U.S. Patent Publication No. 2022/0061678, titled “Detection of Patient Conditions Using Signals Sensed On or Near the Head,” the entire contents of which are incorporated herein by reference. In some examples, IMD 10B may be implanted on other locations of the head or neck, such as a temporal or frontal location of the head.

Although described primarily in the context of examples in which IMDs 10 take the form of an ICM and a cranial sensor device implanted subcutaneously on the back or side of the head or neck, the techniques of this disclosure may be implemented in systems including any one or more implantable or external medical devices, including monitors, pacemakers, defibrillators (e.g., subcutaneous or substernal), wearable external defibrillators (WAEDs), neurostimulators, drug pumps, patch monitors, or wearable physiological monitors, e.g., wrist or head wearable devices. Examples with multiple IMDs or other sensing devices may be able to collect different data useable by system 2 to determine a mental state of patient 4. Furthermore, a system with two devices may capture different values of a common patient parameter with different resolution/accuracy based on their respective locations.

Patient computing devices 12 are configured for wireless communication with IMD 10. Computing devices 12 retrieve event data and other sensed physiological data from IMD 10 that was collected and stored by the IMD. In some examples, computing devices 12 take the form of personal computing devices of patient 4. For example, computing device 12A may take the form of a smartphone of patient 4, and computing device 12B may take the form of a smartwatch or other smart apparel of patient 4. In some examples, computing devices 12 may be any computing device configured for wireless communication with IMD 10, such as a desktop, laptop, or tablet computer. Computing devices 12 may communicate with IMD 10 and each other according to the Bluetooth® or Bluetooth® Low Energy (BLE) protocols, as examples. In some examples, only one of computing devices 12, e.g., computing device 12A, is configured for communication with IMD 10, e.g., due to execution of software (e.g., part of a health monitoring application as described herein) enabling communication and interaction with an IMD.

In some examples, computing device(s) 12, e.g., wearable computing device 12B in the example illustrated by FIG. 1, may include electrodes and other sensors to sense physiological signals of patient 4, and may collect and store physiological data and detect episodes based on such signals. Computing device 12B may be incorporated into the apparel of patient 14, such as within clothing, shoes, eyeglasses, a watch or wristband, a hat, etc. In some examples, computing device 12B is a smartwatch or other accessory or peripheral for a smartphone computing device 12A.

One or more of computing devices 12 may be configured to communicate with a variety of other devices or systems via a network 16. For example, one or more of computing devices 12 may be configured to communicate with one or more computing systems, e.g., computing systems 20A and 20B (collectively, “computing systems 20”) via network 16. Computing systems 20A may be managed by a manufacturer of IMDs 10 to, for example, provide cloud storage and analysis of collected data, maintenance and software services, or other networked functionality for their respective devices and users thereof. Computing system 20A may comprise, or may be implemented by, the Medtronic CareLink™ Network, in some examples. In the example illustrated by FIG. 1, computing system 20A implements a health monitoring system (HMS) 22, although in other examples, either of both of computing systems 20 may implement HMS 22. As will be described in greater detail below, HMS 22 may facilitate determinations of mental status of patient 4 by system 2, and the responsive communication of system 2 to one or more users.

Computing device(s) 12 may transmit data, including data retrieved from IMD(s) 10, to computing system(s) 20 via network 16. The data may include sensed data, e.g., values of physiological parameters measured by IMD(s) 10 and, in some cases one or more of computing devices 12, data regarding determination of mental states by IMD(s) 10 and/or computing device(s) 12, and other physiological signals or data recorded by IMD(s) 10 and/or computing device(s) 12. HMS 22 may also retrieve data regarding patient 4 from one or more sources of electronic health records (EHR) 24 via network. EHR 24 may include data regarding historical (e.g., baseline) physiological parameter values, previous health events and treatments, disease states, comorbidities, demographics, height, weight, and body mass index (BMI), as examples, of patients including patient 4. HMS 22 may use data from EHR 24 to configure algorithms implemented by IMD 10 and/or computing devices 12 to determine mental states for patient 4. In some examples, HMS 22 provides data from EHR 24 to computing device(s) 12 and/or IMD 10 for storage therein and use as part of their algorithms for determining patient mental states.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other but isolates some of the data flows from devices external to the private network for security purposes. In some examples, the communications between the computing devices and systems illustrated in FIG. 1 are encrypted.

Environment 28 of patient 4 may be a home, office, or place of business, or public venue, as examples. Environment 28 may include one or more Internet of Things (IoT) devices, such as IoT devices 30A-30D (collectively “IoT devices 30”) illustrated in the example of FIG. 1. IoT devices 30 may include, as examples, so called “smart” speakers, cameras, televisions, lights, locks, thermostats, appliances, actuators, controllers, or any other smart home (or building) devices. In the example of FIG. 1, IoT device 30C is a smart speaker and/or controller, which may include a display. Computing device(s) 12 may be configured to wirelessly communicate with IoT devices 30. In some examples, HMS 22 communicates with IoT devices 30 via network 16. In some examples, IMDs 10 are configured to communicate wirelessly with one or more of IoT devices 30. In some examples, IoT device(s) 30 may be configured to provide some or all of the functionality ascribed to computing devices 12 herein. In some examples, IoT device(s) 30 may be configured to collect parameter data of patient 4 for determining the mental state of the patient.

Environment 28 includes computing facilities, e.g., a local network 32, by which computing devices 12, IoT devices 30, and other devices within environment 28 may communicate via network 16, e.g., with HMS 22. For example, environment 28 may be configured with wireless technology, such as IEEE 802.11 wireless networks, IEEE 802.15 ZigBee networks, an ultra-wideband protocol, near-field communication, or the like. Environment 28 may include one or more wireless access points, e.g., wireless access points 34A and 34B (collectively, “wireless access points 34”) that provide support for wireless communications throughout environment 28. Additionally or alternatively, e.g., when local network is unavailable, computing devices 12, IoT devices 30, and other devices within environment 28 may be configured to communicate with network 16, e.g., with HMS 22, via a cellular base station 36 and a cellular network.

Users 40 may receive communications regarding a mental state of patient 4, e.g., alerts, from HMS 22 via computing devices 38. Communications may be sent to users 40 for both improving and worsening mental state of patient 4. Users 40 may include, as examples, clinicians, caregivers, family members, and friends of patient 4.

In some examples, one or more of computing device(s) 12 and IoT device(s) 30 may implement an assistant. The event assistant may provide a conversational interface for patient 4 and/or another user to exchange information with the computing device or IoT device. The event assistant may query the user regarding the condition of patient 4. Responses from the user may be used by processing circuitry to determine the mental state of patient 4 or to provide additional information about the mental state or the condition of patient 4 more generally that may improve the efficacy of the responses to and treatment of the mental state of patient 4. The event assistant may use natural language processing and context data to interpret utterances by the user. In some examples, in addition to receiving responses to queries posed by the assistant, the event assistant may be configured to respond to queries posed by the user.

In some examples, computing device(s) 12 and/or HMS 22 may implement one or more techniques to evaluate the physiological signals sensed by IMD(s) 10 and/or the parameter data determined from the sensed physiological signals, and in some cases additional physiological or other patient parameter data sensed or otherwise collected by the computing device(s) 12 or IoT devices 30, to determine the mental state of the patient. In some examples, computing device(s) 12 and/or computing system(s) 20 may have greater processing capacity than IMD(s) 10, enabling more complex analysis of the data. In some examples, the computing device(s) 12 and/or HMS 22 may apply the data to one or more machine learning models or other artificial intelligence developed algorithms to determine the mental state of patient 4.

Any of IMD(s) 10, computing device(s) 12, IoT device(s) 30, computing device(s) 38 and 42, or HMS 22 may, individually or in any combination, perform the operations described herein for determining a mental state of patient 4 based on at least one of the sensed physiological signals or parameter data. Computing system 20B may be associated with an emergency medical service or other community or medical service for responding to events of patient 4. In some examples, computing system 20B may receive communications regarding determined mental states of patient 4.

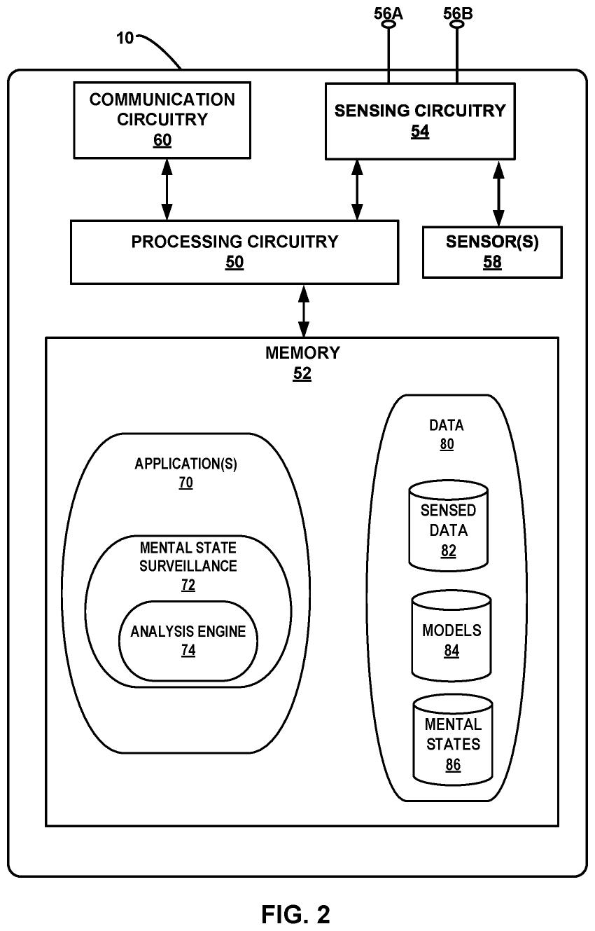

FIG. 2 is a block diagram illustrating an example configuration of an IMD 10 of FIG. 1. As shown in FIG. 2, IMD 10 includes processing circuitry 50, memory 52, sensing circuitry 54 coupled to electrodes 56A and 56B (hereinafter, “electrodes 56”) and one or more sensor(s) 58, and communication circuitry 60.

Processing circuitry 50 may include fixed function circuitry and/or programmable processing circuitry. Processing circuitry 50 may include any one or more of a microprocessor, a controller, a graphics processing unit (GPU), a tensor processing unit (TPU), a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 53 includes computer-readable instructions that, when executed by processing circuitry 50, cause IMD 10 and processing circuitry 50 to perform various functions attributed herein to IMD 10 and processing circuitry 50. Memory 53 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Sensing circuitry 54 may monitor signals from electrodes 56 in order to, for example, monitor electrical activity of a heart of patient 4 and produce an electrocardiogram (ECG) and corresponding ECG data for patient 4, and/or monitor electrical activity of a brain of patient 4 and produce an electroencephalogram (EEG) and corresponding EEG data for patient 4. In some examples, processing circuitry 50 may identify features of the sensed ECG, such as heart rate, heart rate variability, T-wave alternans, intra-beat intervals (e.g., QT intervals), and/or ECG morphologic features. In some examples, processing circuitry 50 may identify features of the sensed EEG collected from one or more locations on the head during one or more physical activity states (e.g., at rest, during activities of daily living, or during sleep)such as increased or decreased activity in one or more frequency bands, e.g., delta (0.5-4 Hz), theta (4-8 Hz), alpha (8-14 Hz), beta (14-30 Hz), or gamma (above 30 Hz), slowing or acceleration of activity in one or more frequency bands, continuity or discontinuity/intermittence of activity in one or more frequency bands, ratio of energy levels of one frequency band to another, frequency shifting across different bands, bispectrum analysis, irregular transitions between different sleep stages, length and frequency of eye movements during sleep, presence of spike and wave complexes, interhemispheric asymmetry, stimulus-triggered evoked potentials, etc. In some examples, sensing circuitry 54 may include a sense amplifier having a bandwidth (e.g., from 0.5 Hz to 200 Hz or greater) sufficient for sensing the EEG and identifying features in each the identified frequency bands.

In some examples, sensing circuitry 54 measures impedance, e.g., of tissue proximate to IMD 10, via electrodes 56. The measured impedance may vary based on respiration, cardiac pulse or flow, galvanic skin response, and a degree of perfusion or edema of tissue proximate to electrodes of IMD, e.g., subcutaneous tissue. Processing circuitry 50 may determine physiological data relating to respiration, cardiac pulse or flow, perfusion, galvanic skin response, and/or edema based on the measured impedance.

In some examples, IMD 10 includes one or more sensors 58, such as one or more accelerometers, gyroscopes, microphones or other sound sensors, optical sensors, temperature sensors, pressure sensors, and/or chemical sensors. In some examples, sensing circuitry 52 may include one or more filters and amplifiers for filtering and amplifying physiological signals received from one or more of electrodes 56 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological parameter data, e.g., values of physiological parameters of patient 4, based on signals from sensors 58, which may be stored in memory 52. Patient parameters determined from signals from sensors 58 may include oxygen saturation, glucose level, stress hormone level, heart sounds, body motion, body posture, blood pressure, respiration, respiration rate, respiration effort, respiration patterns (e.g., associated with sobbing, coughing, or snoring) and/or voice characteristics. For examples, processing circuitry 50 may identify crying/sobbing episodes based on one or more physiological signals such as a respiration or motion signal. Processing circuitry 50 may determine characteristics of such episodes, e.g., duration, intensity, frequency, and determine the mental state of patient 4 based on such characteristics. Based on a sound or vibration sensor, processing circuitry 50 may monitor mechanical function of the heart of patient 4 and produce acoustic heart sounds (HS), or monitor patient's speech/talking, verbal social contacts, etc., which may provide orthogonal information in addition to EEG and ECG signals.

Memory 52 may store applications 70 executable by processing circuitry 50, and data 80. Applications 70 may include a mental state surveillance application 72. Processing circuitry 50 may execute mental state surveillance application 72 to determine a mental state of patient 4 based on combination of one or more of the types of physiological signals/data described herein, which may be stored as sensed data 82. In some examples, sensed data 82 may additionally include patient parameter data sensed by other devices, e.g., computing device(s) 12 or IoT device(s) 30 and received via communication circuitry 60. Mental state surveillance application 72 may be configured with an analysis engine 74. Analysis engine 74 may apply models 84 to sensed data 82 to determine the mental state of patient 4. Models 84 may include one or more rules, algorithms, decision trees, and/or thresholds. In some cases, models 84 may be developed based on machine learning, e.g., may include one or more machine learning models.

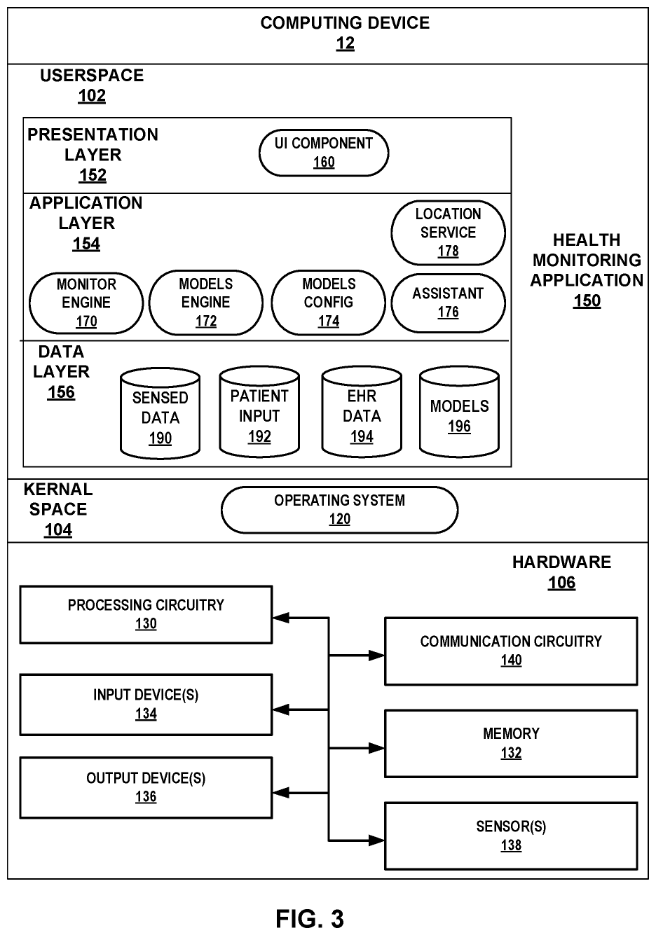

FIG. 3 is a block diagram illustrating an example configuration of a computing device 12 of patient 4, which may correspond to either (or both operating in coordination) of computing devices 12A and 12B illustrated in FIG. 1. In some examples, computing device 12 takes the form of a smartphone, a laptop, a tablet computer, a personal digital assistant (PDA), a smartwatch or other wearable computing device. In some examples, IoT devices 30 and/or computing devices 38 and 42 may be configured similarly to the configuration of computing device 12 illustrated in FIG. 3.

As shown in the example of FIG. 3, computing device 12 may be logically divided into user space 102, kernel space 104, and hardware 106. Hardware 106 may include one or more hardware components that provide an operating environment for components executing in user space 102 and kernel space 104. User space 102 and kernel space 104 may represent different sections or segmentations of memory, where kernel space 104 provides higher privileges to processes and threads than user space 102. For instance, kernel space 104 may include operating system 120, which operates with higher privileges than components executing in user space 102.

As shown in FIG. 3, hardware 106 includes processing circuitry 130, memory 132, one or more input devices 134, one or more output devices 136, one or more sensors 138, and communication circuitry 140. Although shown in FIG. 3 as a stand-alone device for purposes of example, computing device 12 may be any component or system that includes processing circuitry or other suitable computing environment for executing software instructions and, for example, need not necessarily include one or more elements shown in FIG. 3.

Processing circuitry 130 is configured to implement functionality and/or process instructions for execution within computing device 12. For example, processing circuitry 130 may be configured to receive and process instructions stored in memory 132 that provide functionality of components included in kernel space 104 and user space 102 to perform one or more operations in accordance with techniques of this disclosure. Examples of processing circuitry 130 may include, any one or more microprocessors, controllers, GPUs, TPUs, DSPs, ASICs, FPGAs, or equivalent discrete or integrated logic circuitry.

Memory 132 may be configured to store information within computing device 12, for processing during operation of computing device 12. Memory 132, in some examples, is described as a computer-readable storage medium. In some examples, memory 132 includes a temporary memory or a volatile memory. Examples of volatile memories include random access memories (RAM), dynamic random access memories (DRAM), static random access memories (SRAM), and other forms of volatile memories known in the art. Memory 132, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or electrically erasable and programmable (EEPROM) memories. In some examples, memory 132 includes cloud-associated storage.

One or more input devices 134 of computing device 12 may receive input, e.g., from patient 4 or another user. Examples of input are tactile, audio, kinetic, and optical input. Input devices 134 may include, as examples, a mouse, keyboard, voice responsive system, camera, buttons, control pad, microphone, presence-sensitive or touch-sensitive component (e.g., screen), or any other device for detecting input from a user or a machine.

One or more output devices 136 of computing device 12 may generate output, e.g., to patient 4 or another user. Examples of output are tactile, haptic, audio, and visual output. Output devices 134 of computing device 12 may include a presence-sensitive screen, sound card, video graphics adapter card, speaker, cathode ray tube (CRT) monitor, liquid crystal display (LCD), light emitting diodes (LEDs), or any type of device for generating tactile, audio, and/or visual output.

One or more sensors 138 of computing device 12 may sense physiological parameters or signals of patient 4. Sensor(s) 138 may include ECG electrodes, EEG electrodes, accelerometers (e.g., 3-axis accelerometers), optical sensors, EMG sensors, impedance sensors, temperature sensors, pressure sensors, heart sound sensors (e.g., microphones), chemical sensors, and other sensors, and sensing circuitry (e.g., including an ADC), similar to those described above with respect to IMD 10 and FIG. 2.

Communication circuitry 140 of computing device 12 may communicate with other devices by transmitting and receiving data. Communication circuitry 140 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, Wi-Fi (e.g., 802.11 or 802.15 ZigBee), Bluetooth®, or Bluetooth® Low Energy (BLE).

As shown in FIG. 3, health monitoring application 150 executes in user space 102 of computing device 12. Health monitoring application 150 may be logically divided into presentation layer 152, application layer 154, and data layer 156. Presentation layer 152 may include a user interface (UI) component 160, which generates and renders user interfaces of health monitoring application 150.

Application layer 154 may include, but is not limited to, a monitoring engine 170, models engine 172, models configuration component 174, assistant 176, and location service 178. Monitoring engine 172 may be responsive to receipt of a parameter data and physiological signals from IMD 10 and/or IoT devices 30. Monitoring engine 172 may control performance of any of the operations in response to such data ascribed herein to computing device 12, such as analyzing data, determining mental states, transmitting messages to HMS 22.

Monitoring engine 174 analyzes sensed data 190, and in some examples, patient input 192 and/or EHR data 194, to determine a mental state of patient 4. Sensed data 190 may include data received from IMD 10 and physiological and other data related to the condition of patient 4 collected by, for example, computing device(s) 12 and/or IoT devices 30. As examples, sensed data 190 from computing device(s) 12 and/or IoT devices 30 may include one or more of: activity levels, walking/running distance, resting energy, active energy, exercise minutes, quantifications of standing, body mass, body mass index, heart rate, low, high, and/or irregular heart rate events, heart rate variability, EEG band activity, sleep stages, walking heart rate, heart beat series, digitized ECG, blood oxygen saturation, blood pressure (systolic and/or diastolic), respiratory rate, respiratory effort, maximum volume of oxygen, blood glucose, peripheral perfusion, galvanic skin response, movement, e.g., within an environment of an IoT device, sleep patterns, or any other signals/parameters described herein.

Patient input 192 may include responses to queries posed by health monitoring application 150 regarding the condition of patient 4, input by patient 4 or another user, such as bystander 26. The queries and responses may occur responsive to the detection of the event by IMD 10, or may have occurred prior to the detection, e.g., as part long-term monitoring of the health of patient 4. User recorded health data may include one or more of: exercise and activity data, sleep data, symptom data, medical history data, quality of life data, nutrition data, medication taking or compliance data, allergy data, demographic data, weight, and height. EHR data 194 may include any of the information regarding the historical condition (e.g., comorbid conditions) or treatments of patient 4 described above. EHR data 194 may include demographic and other information of patient 4, such as age, gender, race, height, weight, and BMI.

Monitoring engine 172 may apply models 196 to the data. Models 196 may include one or more rules, algorithms, decision trees, and/or thresholds. In some cases, models 196 may be developed based on machine learning, e.g., may include one or more machine learning models. In some examples, models 196 and the operation of models/rules engine 172 may provide a more complex analysis the patient parameter data, e.g., the data received from IMD 10. In examples in which models 196 include one or more machine learning models, monitoring engine 172 may apply raw data, e.g., signal and parameter data, or feature vectors derived from the signals/data to the model(s).

Models configuration component 174 may be configured to modify models 196 (and in some examples models 84) based on feedback indicating whether the determined mental states were accurate. The feedback may be received from patient 4, or from care providers 40 and/or EHR 24 via HMS 22. Models configuration component 174, or another component executed by processing circuitry of system 2, may select a configuration of models 196 based on etiological data for patient, e.g., any combination of one or more of the examples of sensed data 190, patient input 192, and EHR data 194 discussed above. In some examples, different sets of models 196 tailored to different cohorts of patients may be available for selection for patient 4 based on such etiological data.

As discussed above, assistant 176 may provide a conversational interface for patient 4 to exchange information with computing device 12. Assistant 176 may query the user regarding the condition of patient 4. Responses from the user may be included as patient input 192. Assistant 176 may use natural language processing and context data to interpret utterances by the user. In some examples, in addition to receiving responses to queries posed by the assistant, assistant 176 may be configured to respond to queries posed by the user, or to receive general spoken input regarding patient condition.

Location service 178 may determine the location of computing device 12 and, thereby, the presumed location of patient 4. Location service 178 may use global position system (GPS) data, multilateration, and/or any other known techniques for locating computing devices. Processing circuitry 130 may store locations of patient 4 over time determined by location service 178 in memory 132. Monitor engine 170 may determine mental states of patient 4 based on the locations, e.g., based on deviations from periodic (e.g., daily) movement patterns of patient, and or based on movement within similar or shorter periods of time being above or below a threshold, e.g., determined based on a baseline for the patient.

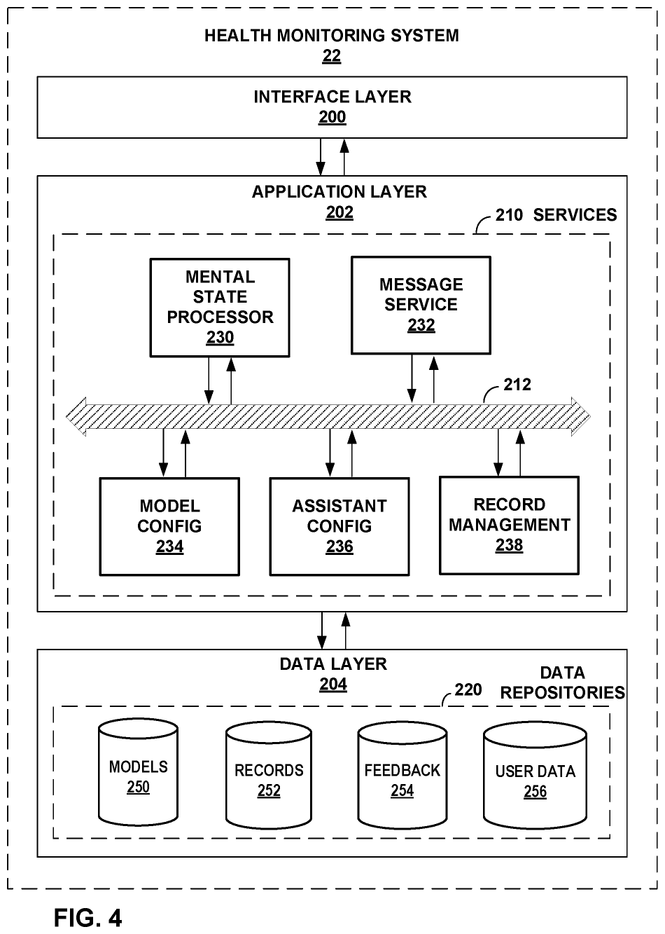

FIG. 4 is a block diagram illustrating an operating perspective of HMS 22. HMS 22 may be implemented in a computing system 20, which may include hardware components such as those of computing device 12, e.g., processing circuitry, memory, and communication circuitry, embodied in one or more physical devices. FIG. 4 provides an operating perspective of HMS 22 when hosted as a cloud-based platform or cloud computing system. In the example of FIG. 4, components of HMS 22 are arranged according to multiple logical layers that implement the techniques of this disclosure. Each layer may be implemented by one or more modules comprised of hardware, software, or a combination of hardware and software.

Computing devices, such as computing devices 12, IoT devices 30, computing devices 38, and computing device 42, operate as clients that communicate with HMS 22 via interface layer 200. The computing devices typically execute client software applications, such as desktop application, mobile application, and web applications. Interface layer 200 represents a set of application programming interfaces (API) or protocol interfaces presented and supported by HMS 22 for the client software applications. Interface layer 200 may be implemented with one or more web servers.

As shown in FIG. 4, HMS 22 also includes an application layer 202 that represents a collection of services 210 for implementing the functionality ascribed to HMS herein.

Application layer 202 receives information from client applications, e.g., a determined mental state from an IMD 10 and/or a computing device 12, and further processes the information according to one or more of the services 210 to respond to the information. Application layer 202 may be implemented as one or more discrete software services 210 executing on one or more application servers, e.g., physical or virtual machines. That is, the application servers provide runtime environments for execution of services 210. In some examples, the functionality interface layer 200 as described above and the functionality of application layer 202 may be implemented at the same server. Services 210 may communicate via a logical service bus 212. Service bus 212 generally represents a logical interconnection or set of interfaces that allows different services 210 to send messages to other services, such as by a publish/subscription communication model.

Data layer 204 of HMS 22 provides persistence for information in PPEMS 6 using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

As shown in FIG. 4, each of services 230-238 is implemented in a modular form within HMS 22. Although shown as separate modules for each service, in some examples the functionality of two or more services may be combined into a single module or component. Each of services 230-238 may be implemented in software, hardware, or a combination of hardware and software. Moreover, services 230-238 may be implemented as standalone devices, separate virtual machines or containers, processes, threads, or software instructions generally for execution on one or more physical processors.

Mental state processor service 230 may be responsive to receipt of physiological signals and/or parameter data from other components of system 2 to determine mental states of patient 4 as described herein or may be responsive to mental states determined by other components of system 2 to facilitate communication, e.g., to patient 4 or other users 40.

Record management service 238 may store the patient data within records 252. Message service 232 may package some or all of the data from the record, in some cases with additional information as described herein, into one or more messages for transmission to patient 4 and/or users 40. User data 256 may store data used by message service 232 to identify to whom to send messages based on preferences of patient 4.

In examples in which HMS 22 performs an analysis to determine mental states based on parameter data of patient 4, processor service 230 may apply one or more models 250 to the data received, e.g., to feature vectors derived by event processor service 230 from the data, or to raw data, e.g., digitized ECG, EEG, or other waveforms. Models 250 may include one or more rules, algorithms, decision trees, and/or thresholds, or machine learning models which may be developed by model configuration service 234 based on machine learning. Example machine learning techniques that may be employed to generate models 250 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbor (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, Least-Angle Regression (LARS), Principal Component Analysis (PCA) and Principal Component Regression (PCR).

Model configuration service 234 may be configured to modify these models/rules based on feedback data 254 that indicates whether the determined mental states were accurate. Feedback 254 may be received from patient 4, e.g., via computing device(s) 12, or from users 40 and/or EHR 24.

As illustrated in the example of FIG. 4, services 210 may also include an assistant configuration service 236 for configuring and interacting with assistant 176 implemented in computing device 12 or other computing devices. For example, assistant configuration service 236 may provide assistants updates to their natural language processing and context analyses to improve their operation over time.

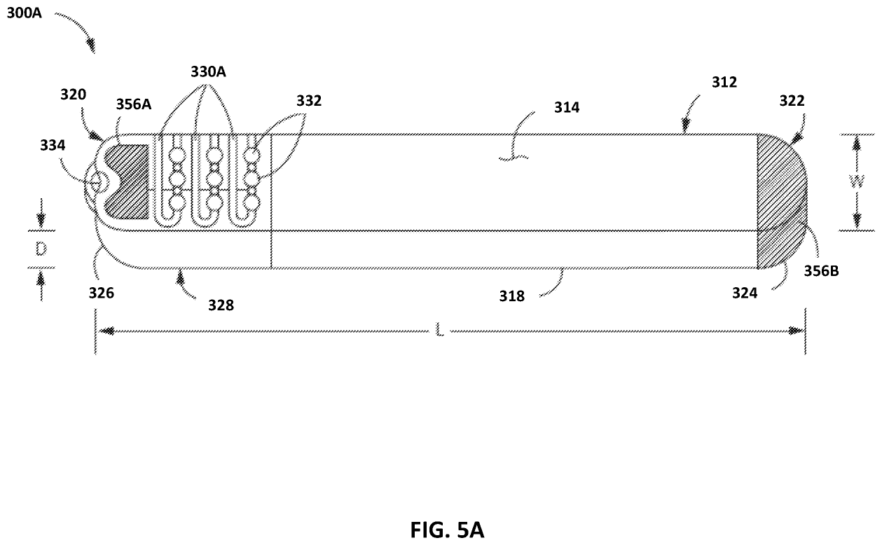

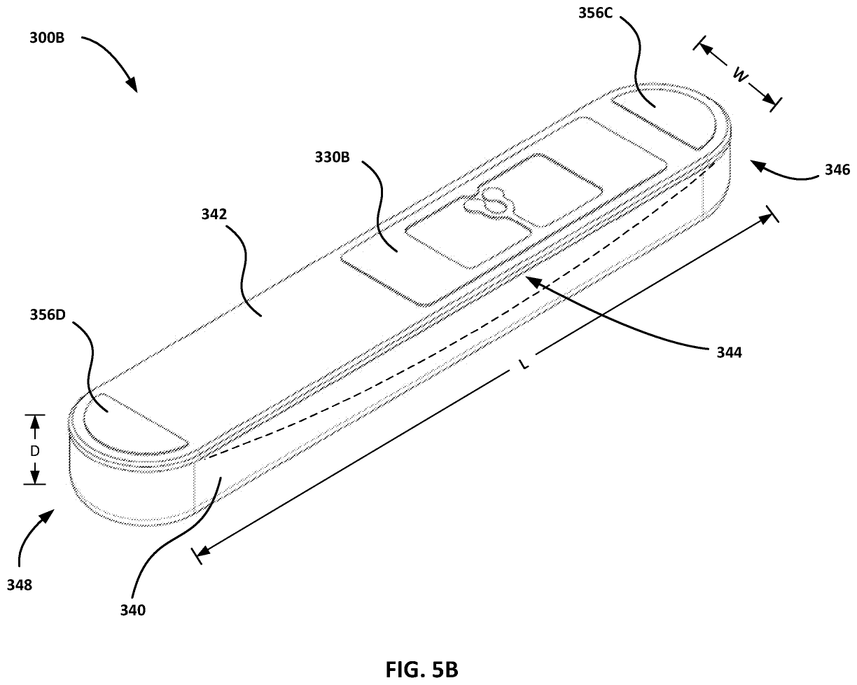

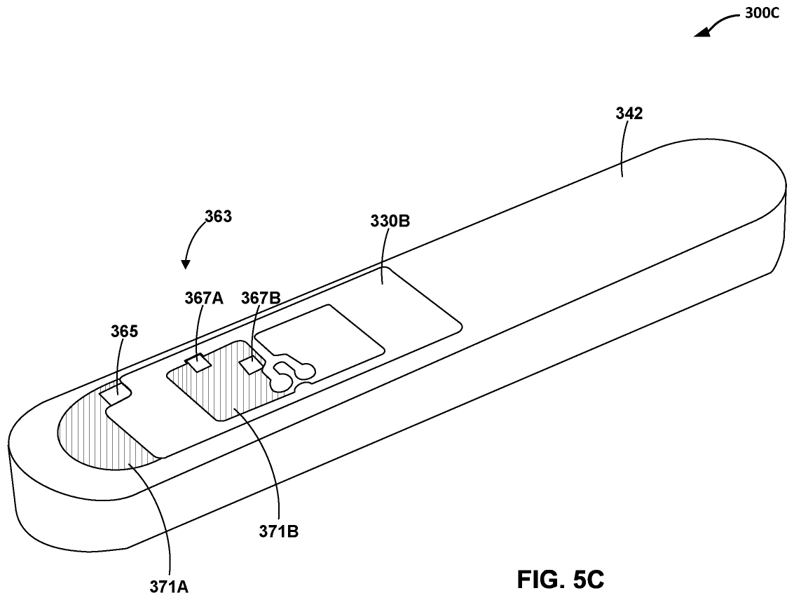

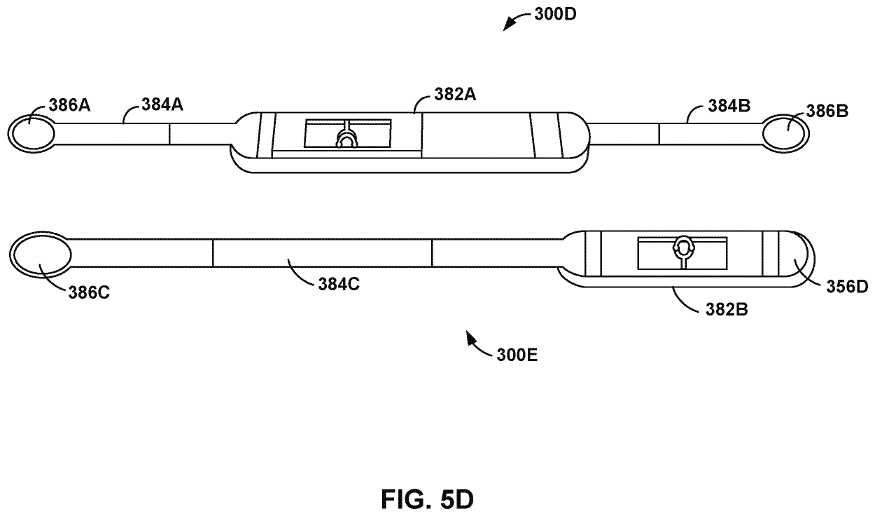

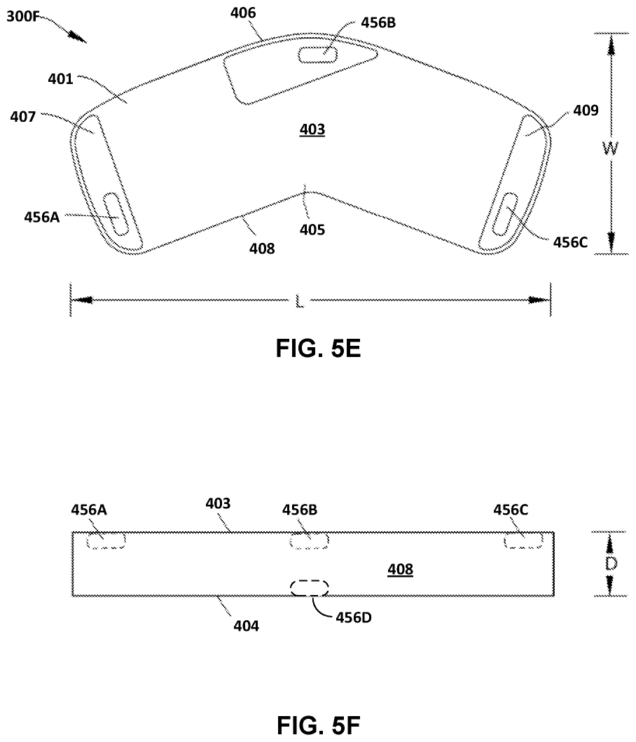

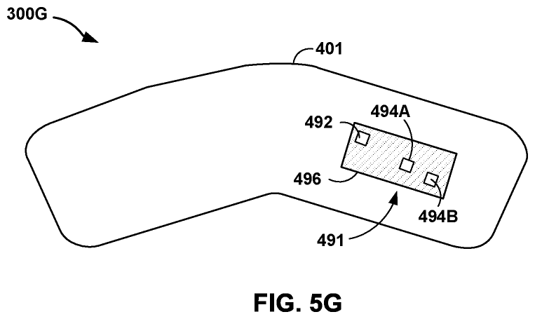

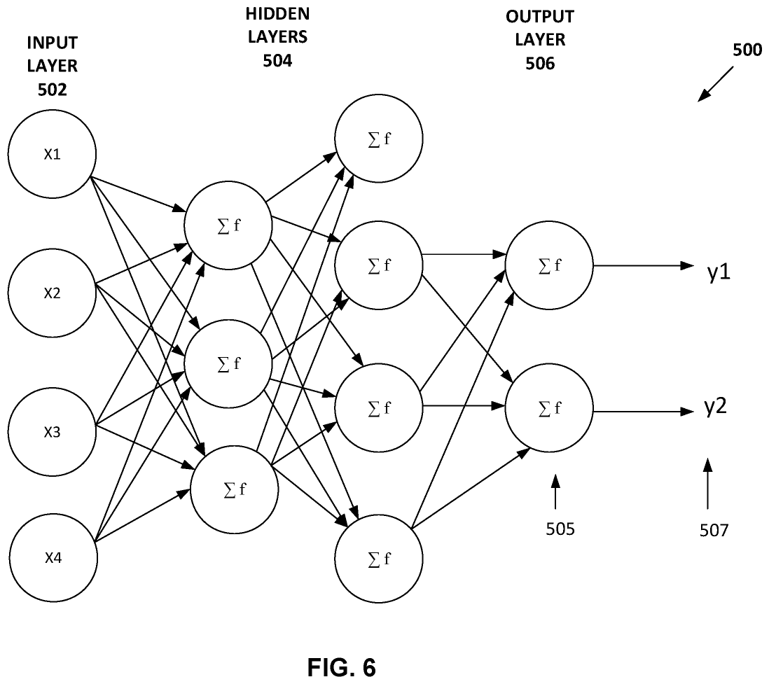

FIGS. 5A-5G are conceptual diagrams illustrating example implantable monitoring devices. For example, FIG. 5A is a perspective drawing illustrating an IMD 300A, which may be an example configuration of IMD 10A or IMD 10B of FIG. 1 as an ICM. In the example shown in FIG. 5A, IMD 300A may be embodied as a monitoring device having housing 312, proximal electrode 356A and distal electrode 356B. Housing 312 may further comprise first major surface 314, second major surface 318, proximal end 320, and distal end 322. Housing 312 encloses electronic circuitry located inside the IMD 300A, e.g., such as described with respect to IMD 10 and FIG. 2, and protects the circuitry contained therein from body fluids. Housing 12 may be hermetically sealed and configured for subcutaneous implantation. Electrical feedthroughs provide electrical connection of electrodes 356A and 356B.

In the example shown in FIG. 5A, IMD 300A is defined by a length L, a width W and thickness or depth D and is in the form of an elongated rectangular prism wherein the length L is much larger than the width W, which in turn is larger than the depth D. In one example, the geometry of the IMD 300A—in particular, a width W greater than the depth D—is selected to allow IMD 300A to be inserted under the skin of the patient using a minimally invasive procedure and to remain in the desired orientation during insertion. For example, the device shown in FIG. 5A includes radial asymmetries (notably, the rectangular shape) along the longitudinal axis that maintains the device in the proper orientation following insertion. For example, the spacing between proximal electrode 356A and distal electrode 356B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 300A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 314 may range from 3 mm to 15, mm, from 3 mm to 10 mm, or from 5 mm to 15 mm, and may be any single or range of widths between 3 mm and 15 mm. The thickness of depth D of IMD 300A may range from 2 mm to 15 mm, from 2 mm to 9 mm, from 2 mm to 5 mm, from 5 mm to 15 mm, and may be any single or range of depths between 2 mm and 15 mm. In addition, IMD 300A, according to an example of the present disclosure, has a geometry and size designed for ease of implant and patient comfort. Examples of IMD 300A described in this disclosure may have a volume of three cubic centimeters (cm) or less, 1.5 cubic cm or less or any volume between three and 1.5 cubic centimeters.

In the example shown in FIG. 5A, once inserted within the patient, the first major surface 314 faces outward, toward the skin of the patient while the second major surface 318 is located opposite the first major surface 314. In addition, in the example shown in FIG. 5A, proximal end 320 and distal end 322 are rounded to reduce discomfort and irritation to surrounding tissue once inserted under the skin of the patient. IMD 300A, including instrument and method for inserting IMD 300A is described, for example, in U.S. Patent Publication No. 2014/0276928, incorporated herein by reference in its entirety.

Proximal electrode 356A is at or proximate to proximal end 320, and distal electrode 356B is at or proximate to distal end 322. Proximal electrode 356A and distal electrode 356B are used to sense ECG signals, thoracically outside the ribcage, which may be implanted sub-muscularly or subcutaneously. ECG signals may be stored in a memory of IMD 300A, and data may be transmitted via integrated antenna 330A to another device, which may be another implantable device or an external device, such as a computing device 12. In some examples, electrodes 356A and 356B may additionally or alternatively be used for sensing any biopotential signal of interest, which may be, for example, an EGM, an EEG, an EMG, a nerve signal, or a measure of impedance from any implanted location, e.g., a cranial (back, front, top, temporal) or neck location for EEG sensing. Electrodes 356A and 356B may correspond to electrodes 56A and 56B of FIG. 2.

In the example shown in FIG. 5A, proximal electrode 356A is at or in close proximity to the proximal end 320, and distal electrode 356B is at or in close proximity to distal end 322. In this example, distal electrode 356B is not limited to a flattened, outward facing surface, but may extend from first major surface 314 around rounded edges 324 and/or end surface 326 and onto the second major surface 318 so that the electrode 356B has a three-dimensional curved configuration. In some examples, electrode 356B is an uninsulated portion of a metallic, e.g., titanium, part of housing 312.

In the example shown in FIG. 5A, proximal electrode 356A is located on first major surface 314 and is substantially flat and outward facing. However, in other examples proximal electrode 356A may utilize the three dimensional curved configuration of distal electrode 356B, providing a three dimensional proximal electrode (not shown in this example). Similarly, in other examples distal electrode 356B may utilize a substantially flat, outward facing electrode located on first major surface 314 similar to that shown with respect to proximal electrode 356A.

The various electrode configurations allow for configurations in which proximal electrode 356A and distal electrode 356B are located on both first major surface 314 and second major surface 318. In other configurations, such as that shown in FIG. 5A, only one of proximal electrode 356A and distal electrode 356B is located on both major surfaces 314 and 318, and in still other configurations both proximal electrode 356A and distal electrode 356B are located on one of the first major surface 314 or the second major surface 318 (e.g., proximal electrode 356A located on first major surface 314 while distal electrode 356B is located on second major surface 318). In another example, IMD 300A may include electrodes on both major surface 314 and 318 at or near the proximal and distal ends of the device, such that a total of four electrodes are included on IMD 300A. Electrodes 356A and 356B may be formed of a plurality of different types of biocompatible conductive material, e.g., stainless steel, titanium, platinum, iridium, or alloys thereof, and may utilize one or more coatings such as titanium nitride or fractal titanium nitride.

In the example shown in FIG. 5A, proximal end 320 includes a header assembly 328 that includes one or more of proximal electrode 356A, integrated antenna 330A, anti-migration projections 332, and/or suture hole 334. Integrated antenna 330A is located on the same major surface (i.e., first major surface 314) as proximal electrode 356A and is also included as part of header assembly 328. Integrated antenna 330A allows IMD 300A to transmit and/or receive data. In other examples, integrated antenna 330A may be formed on the opposite major surface as proximal electrode 356A or may be incorporated within the housing 312 of IMD 10A. In the example shown in FIG. 5A, anti-migration projections 332 are located adjacent to integrated antenna 330A and protrude away from first major surface 314 to prevent longitudinal movement of the device. In the example shown in FIG. 5A, anti-migration projections 332 include a plurality (e.g., nine) small bumps or protrusions extending away from first major surface 314. As discussed above, in other examples, anti-migration projections 332 may be located on the opposite major surface as proximal electrode 356A and/or integrated antenna 330A. In addition, in the example shown in FIG. 5A, header assembly 328 includes suture hole 334, which provides another means of securing IMD 300A to the patient to prevent movement following insertion. In the example shown, suture hole 334 is located adjacent to proximal electrode 356A. In one example, header assembly 328 is a molded header assembly made from a polymeric or plastic material, which may be integrated or separable from the main portion of IMD 300A.

FIG. 5B is a perspective drawing illustrating another IMD 300B, which may be another example configuration of IMD 10A or IMD 10B from FIG. 1 as an ICM. IMD 300B of FIG. 5B may be configured substantially similarly to IMD 300A of FIG. 5A, with differences between them discussed herein.

IMD 300B may include a leadless, subcutaneously-implantable monitoring device, e.g. an ICM. IMD 300B includes housing having a base 340 and an insulative cover 342. Proximal electrode 356C and distal electrode 356D may be formed or placed on an outer surface of cover 342. Various circuitries and components of IMD 300B, e.g., described above with respect to FIG. 2, may be formed or placed on an inner surface of cover 342, or within base 340. In some examples, a battery or other power source of IMD 300B may be included within base 340. In the illustrated example, antenna 330B is formed or placed on the outer surface of cover 342 but may be formed or placed on the inner surface in some examples. In some examples, insulative cover 342 may be positioned over an open base 340 such that base 340 and cover 342 enclose the circuitries and other components and protect them from fluids such as body fluids. The housing including base 340 and insulative cover 342 may be hermetically sealed and configured for subcutaneous implantation.

Circuitries and components may be formed on the inner side of insulative cover 342, such as by using flip-chip technology. Insulative cover 342 may be flipped onto a base 340. When flipped and placed onto base 340, the components of IMD 300B formed on the inner side of insulative cover 342 may be positioned in a gap 344 defined by base 340. Electrodes 356C and 356D and antenna 330B may be electrically connected to circuitry formed on the inner side of insulative cover 342 through one or more vias (not shown) formed through insulative cover 342. Insulative cover 342 may be formed of sapphire (i.e., corundum), glass, parylene, and/or any other suitable insulating material. Base 340 may be formed from titanium or any other suitable material (e.g., a biocompatible material). Electrodes 356C and 356D may be formed from any of stainless steel, titanium, platinum, iridium, or alloys thereof. In addition, electrodes 356C and 356D may be coated with a material such as titanium nitride or fractal titanium nitride, although other suitable materials and coatings for such electrodes may be used. Electrodes 356C and 356D may correspond to electrodes 56A and 56B in FIG. 2.

In the example shown in FIG. 5B, the housing of IMD 300B defines a length L, a width W and thickness or depth D and is in the form of an elongated rectangular prism wherein the length L is much larger than the width W, which in turn is larger than the depth D, similar to IMD 300A of FIG. 5A. For example, the spacing between proximal electrode 356C and distal electrode 356D may range from 5 mm to 50 mm, from 30 mm to 50 mm, from 35 mm to 45 mm, and may be any single spacing or range of spacings from 5 mm to 50 mm, such as approximately 40 mm. In addition, IMD 300B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 300B may range from 2 mm to 15 mm, from 5 mm to 15 mm, or from 3 mm to 5 mm, and may be any single depth or range of depths between 2 mm and 15 mm, such as approximately 4 mm. IMD 300B may have a volume of three cubic centimeters (cm) or less, or 1.5 cubic cm or less, such as approximately 1.4 cubic cm.

In the example shown in FIG. 5B, once inserted subcutaneously within the patient, outer surface of cover 342 faces outward, toward the skin of the patient. In addition, as shown in FIG. 5B, proximal end 346 and distal end 348 are rounded to reduce discomfort and irritation to surrounding tissue once inserted under the skin of the patient. In addition, edges of IMD 300B may be rounded.

FIG. 5C depicts another example IMD 300C, which may be substantially similar to IMD 300B of FIG. 5B, except for the differences noted herein. For example, although not illustrated in FIG. 5C, IMD 300C may include electrodes.

IMD 300C includes an optical sensor 363. Optical sensor 363 may be used to sense oxygen saturation, e.g., SpO₂ or StO₂. As another example, the signal sensed by optical sensor 363 may vary with the pulsatile flow of blood. Peak detection and/or other signal processing techniques may be used to identify heart beats within the optical signal. Processing circuitry may determine heart rate, heart rate variability, and other parameters derivable from a time series of heartbeat detections based on optical signal. The processing circuitry may use optical signal as a surrogate for an ECG signal according to any of the techniques described herein.

In some examples, the processing circuitry may determine pulse transit time (PTT) based on depolarizations detected in an ECG signal and features detected in the optical signal.

PTT may be inversely correlated with, and thus indicative of, blood pressure. PTT may act as a surrogate for blood pressure. In some examples, processing circuitry may determine blood pressure based on a morphological and/or machine learning analysis of the photoplethysmography (PPG) signal from optical sensor 363.

Optical sensor 363 includes one or more light emitters 365 and light detectors 367A and 367B (hereinafter, “light detectors 367”). The numbers of light emitters and detectors illustrated in FIG. 2R is an example, and in other examples an optical sensor may include different numbers of light emitters and/or light detectors. In some examples, a surface 377, e.g., a major surface or portion thereof, of a housing 375 may configured as a window that is transparent or substantially transparent to the light, e.g., wavelengths of light, emitted and detected by optical sensor 363.

Light emitter(s) 365 include a light source, such as one or more light emitting diodes (LEDs) or vertical cavity surface emitting lasers (VCSELs), that may emit light at one or more wavelengths within the visible (VIS) and/or near-infrared (NIR) spectra. For example, light emitter(s) 365 may emit light at one or more of about 660 nanometer (nm), 720 nm, 760 nm, 800 nm, or at any other suitable wavelengths.

In some examples, techniques for determining blood oxygenation, e.g., SpO₂ or StO₂, may include using light emitter(s) 365 to emit light at one or more VIS wavelengths (e.g., approximately 660 nm) and at one or more NIR wavelengths (e.g., approximately 850-890 nm). The combination of VIS and NIR wavelengths may help enable processing circuitry to distinguish oxygenated hemoglobin from deoxygenated hemoglobin, since as hemoglobin becomes less oxygenated, an attenuation of VIS light increases and an attenuation of NIR decreases. By comparing the amount of VIS light detected by light detectors 367 to the amount of NIR light detected by light detectors 367, processing circuitry may determine the relative amounts of oxygenated and deoxygenated hemoglobin in the tissue of a patient.

Techniques for determining a blood oxygenation value or sensing the pulsatile flow of blood using an optical signal may be based on the optical properties of blood-perfused tissue that change depending upon the relative amounts of oxygenated and deoxygenated hemoglobin in the microcirculation of tissue. These optical properties are due, at least in part, to the different optical absorption spectra of oxygenated and deoxygenated hemoglobin. Thus, the oxygen saturation level of the patient's tissue may affect the amount of light that is absorbed by blood within the tissue, and the amount of light that is reflected by the tissue. Light detectors 367 each may receive light from light emitter 365 that is reflected by the tissue and generate electrical signals indicating the intensities of the light detected by light detectors 367. Processing circuitry then may evaluate the electrical signals from light detectors 367 in order to determine an oxygen saturation value, to detect heart beats, and/or to determine PTT values. In some examples, light emitter 365 may additionally or alternatively emit other wavelengths of light, such as green or amber light, because the variation of signals detected by detectors 367 with pulsatile blood flow may be greater at such wavelengths, which may increase the ability to detect pulses to identify heart beats and/or determine PTT.

In some examples, a difference between the electrical signals generated by light detectors 367A and 367B may enhance an accuracy of the determinations. For example, because tissue absorbs some of the light emitted by light emitter 365, the intensity of the light reflected by tissue becomes attenuated as the distance (and amount of tissue) between light emitter 365 and light detectors 367 increases. Thus, because light detector 367B is positioned further from light emitter 365 than light detector 367A, the intensity of light detected by light detector367B should be less than the intensity of light detected by light detector 367A. Due to the close proximity of detectors 367A, 367B to one another, the difference between the intensity of light detected by light detector 367A and the intensity of light detected by light detector 367B should be attributable only to the difference in distance from light emitter 365.

As illustrated in FIG. 5C, IMD 300C includes antenna 330B disposed on cover 342. In some examples, antenna 330B may include a substrate layer and a metalized layer formed on cover 342. The metalized layer may include, for example, aluminum, copper, silver, or other conductive metals. Antenna 330B may include other materials, such as, for example, ceramics or other dielectrics (e.g., as in dielectric resonator antennas). Regardless of the material, antenna 330B may include an opaque or substantially opaque material. For example, an opaque (e.g., or substantially opaque) material may block transmission of at least a portion of radiation of a selected wavelength, such as, between about 75% and about 100% of visible light.

In examples in which antenna 330B includes an opaque material, components of optical sensor 363 may be arranged relative to portions of antenna 330B to reduce or prevent optical interference between components. For example, as illustrated in FIG. 5C, light emitter 365 is positioned on an outer perimeter of antenna 330B, whereas light detectors 367 are positioned within an aperture defined by antenna 330B. In this way, antenna 330B may define an optical boundary of opaque material that reduces or prevents transmission of light from emitter 365 directly to detectors 367. Rather, light emitted from light emitter 365 must travel through tissue. In some examples, one or more optical masks 371A and 371B may be applied to further prevent optical interference.

FIG. 5D is a conceptual diagram illustrating example IMDs 300D and 300E including respective bodies 382A and 382B. Bodies 382A and 382B of IMDs 300D and 300E may include the features ascribed to IMDs in FIGS. 1-5C, such as housings containing circuitry, electrodes, optical sensors, and antennas. As illustrated in FIG. 5D, IMD 300D may include extensions 384A and 384B, and IMD 300E may include extension 384C. Extensions 384A and 384B respectively include electrodes 386A and 386B, and extension 384C includes electrode 386C. In general, extensions 384A, 384B, and 384C (collectively, “extensions 384”) space electrodes 386A, 386B, and 386C (collectively, “electrodes 386”) away from bodies 382A and 382B. Extensions 384 may increase a distance or provide directional flexibility for a vector between electrodes 384 (or between an electrode 384 and an electrode 356D on housing as illustrated for IMD 300E). IMDs 300A and 300B may be used as cranial sensing devices to sense EEG signals.

FIG. 5E depicts a top view of an IMD 300F in accordance with examples of this disclosure. FIG. 5F depicts a side view of IMD 300F shown in FIG. 5E. In some examples, IMD 300F can include some or all the features of, and be similar to, IMDs 10 described above with respect to FIGS. 1 and 2 and/or IMDs 300A-300E described below with respect to FIGS. 5A-5D. In the illustrated example, IMD 300F includes a housing 401 that carries a plurality of electrodes 456A, 456B, 456C, and 456D (collectively “electrodes 456”) thereon. Although four electrodes 456 are shown for IMD 300F, in other examples, only two or three electrodes, or more than four electrodes may be carried by housing 401.

Housing 401 additionally encloses electronic circuitry, e.g., as described above with respect to IMD 10 and FIG. 2, and protects the circuitry contained therein from body fluids. In various examples, electrodes 456 can be disposed along any surface of the sensor device 300F (e.g., anterior surface, posterior surface, left lateral surface, right lateral surface, superior side surface, inferior side surface, or otherwise), and the surface in turn may take any suitable form.

In the example of FIGS. 5E and 5F, housing 401 can be a biocompatible material having a relatively planar shape including a first major surface 403 configured to face towards the tissue of interest (e.g., to face anteriorly when positioned at the back of the patient's neck) a second major surface 404 opposite the first, and a depth D or thickness of housing 401 extending between the first and second major surfaces. Housing 401 can define a superior side surface 406 (e.g., configured to face superiorly when IMD 300F is implanted in or at the patient's head or neck) and an opposing inferior side surface 408. Housing 401 can further include a central portion 405, a first lateral portion (or left portion) 407, and a second lateral portion (or right portion) 409. Electrodes 456 are distributed about housing 401 such that a central electrode 456B is disposed within the central portion 405 (e.g., substantially centrally along a horizontal axis of the device), a back electrode 456D is disposed on inferior side surface 408, a left electrode 456A electrode is disposed within the left portion 407, and a right electrode 456C is disposed within the right portion 409. As illustrated, housing 401 can define a boomerang or chevron-like shape in which the central portion 405 includes a vertex, with the first and second lateral portions 407 and 409 extending both laterally outward and from the central portion 405 and at a downward angle with respect to a horizontal axis of the device. In other examples, housing 401 may be formed in other shapes, which may be determined by desired distances or angles between different electrodes 456 carried by housing 401. In some examples, housing may have a curved shape in the direction of its thickness.

The configuration of housing 401, e.g., as relatively thin, can advantageously facilitate subcutaneous implantation. Additionally, housing 401 can be flexible, so that housing 201 can at least partially bend to correspond to the anatomy of the patient's neck or head (e.g., with left and right lateral portions 407 and 409 of housing 401 bending anteriorly relative to the central portion 405 of housing 401).

In some examples, housing 401 can have a length L of from about 15 to about 50 mm, from about 20 to about 30 mm, or about 25 mm. Housing 401 can have a width W from about 2.5 to about 15 mm, from about 5 to about 10 mm, or about 7.5 mm. In some embodiments, housing 401 can have a thickness less than about 10 mm, about 9 mm, about 8 mm, about 7 mm, about 6 mm, about 5 mm, about 4 mm, or about 3 mm. In some examples, the thickness of housing 401 can be from about 2 to about 8 mm, from about 3 to about 5 mm, or about 4 mm. Housing 401 can have a volume of less than about 1.5 cc, about 1.4 cc, about 1.3 cc, about 1.2 cc, about 1.1 cc, about 1.0 cc, about 0.9 cc, about 0.8 cc, about 0.7 cc, about 0.6 cc, about 0.5 cc, or about 0.4 cc. In some examples, housing 401 can have dimensions suitable for implantation through a trocar introducer or any other suitable implantation technique.

As illustrated, electrodes 456 carried by housing 401 are arranged so that all three electrodes 456 do not lie on a common axis. In such a configuration, electrodes 456 can achieve a variety of signal vectors, which may provide one or more improved signals, as compared to electrodes that are all aligned along a single axis. This can be particularly useful in an IMD configured to be implanted at the neck or head while detecting electrical activity in the brain and the heart, e.g., both an EEG and ECG. In some examples, processing circuitry may create virtual signal vectors through a weighted sum of two or more physical signal vectors, such as the physical signal vectors available from electrodes 456 of IMD 300F or the electrodes of any other implantable monitoring device described herein.

In some examples, all electrodes 456 are located on the first major surface 203 and are substantially flat and outwardly facing. However, in other examples one or more electrodes 456 may utilize a three-dimensional configuration (e.g., curved around an edge of IMD 300F).

Similarly, in other examples, such as that illustrated in FIG. 5F, one or more electrodes 456 may be disposed on the second major surface opposite the first. The various electrode configurations allow for configurations in which electrodes 456 are located on both the first major surface and the second major surface. Electrodes 456 may be formed of a plurality of different types of biocompatible conductive material (e.g., titanium nitride or platinum iridium), and may utilize one or more coatings such as titanium nitride or fractal titanium nitride. In some examples, the material choice for electrodes can also include materials having a high surface area (e.g., to provide better electrode capacitance for better sensitivity) and roughness (e.g., to aid implant stability). Although the example shown in FIGS. 5E and 5F includes four electrodes 456, in some examples IMD 300F can include 1, 2, 3, 4, 5, 6, or more electrodes carried by housing 401.

FIG. 5G depicts an example IMD 300G that includes an optical sensor 491. IMD 300G may otherwise be configured similarly to any of the other IMDs described herein, e.g., such as IMD 300F of FIGS. 5E and 5F. Optical sensor 491 includes a light emitter 492, and light detectors 494A and 494B (hereinafter, “light detectors 294”). Optical sensor 491, light emitter 492, and light detectors 494 may be configured as described above with respect to FIG. 5C and optical sensor 363, optical emitter 365, and optical detectors 367.

In some examples, optical sensor 491 comprises a window 496, e.g., glass or sapphire, formed as a portion of housing 401. Light emitter 492 and light detectors 494 may be located beneath window 496. Window 496 may be transparent or substantially transparent to the light, e.g., wavelengths of light, emitted and detected by optical sensor 491. In some examples, all or a substantial portion of one of the major surfaces of housing 401 may formed as window 496.

In some examples, one or more portions of window 496 may be optically masked. In some examples, portions of window, with the exception of those above emitter 492 and detectors 494 may be optically masked. Optical masking may reduce or prevent transmission of light, e.g., to prevent internal reflection within window 496 that may confound measurements. An optical mask may include a material configured to substantially absorb emitted light, such as titanium nitride, columnar titanium nitride, titanium, or another material suitable to absorb selected wavelengths of light that may be emitted by light emitter 492.

FIG. 6 is a conceptual diagram illustrating an example machine learning model 500 configured to determine a mental state of patient 4 based on one or more physiological signals and/or parameter data collected by system 2, e.g., by IMDs 10, any other implantable monitoring devices, computing devices 12, and/or IoT devices 30. Machine learning model 500 is an example of a deep learning model, or deep learning algorithm, trained to determine a mental state, e.g., from a plurality of mental states as classifications, and/or output one or more scores or index values for one or more mental states, e.g., indicative of a degree of the mood disorder or other mental state, or likelihood of worsening of the mental state. One or more of IMDs 10, 300, computing devices 12, and/or HMS 22 may train, store, and/or utilize machine learning model 500, but other devices may apply inputs associated with a particular patient to machine learning model 500 in other examples. In some examples, machine learning model 500 comprises a convolutional neural network. Some non-limiting examples of models that may be used for determining mental states of patients based on input data including physiological signals/parameters include ResNet-18 may be used AlexNet, VGGNet, GoogleNet, ResNet50, or DenseNet, etc. Some non-limiting examples of machine learning techniques include Support Vector Machines, K-Nearest Neighbor algorithm, and Multi-layer Perceptron.

As shown in the example of FIG. 6, machine learning model 500 may include three layers. These three layers include input layer 502, one or more hidden layers 504, and output layer 506. Output layer 506 comprises the output from the transfer function 505 of output layer 506. Input layer 502 represents each of the input values X1 through X4 provided to machine learning model 500. The input values may be any signals or parameters described herein for determining a mental state of a patient, or features derived therefrom.

Each of the input values for each node in the input layer 502 is provided to each node of hidden layer 504. In the example of FIG. 6, hidden layers 504 include two layers, one layer having four nodes and the other layer having three nodes, but fewer or greater number of nodes may be used in other examples. Each input from input layer 502 is multiplied by a weight and then summed at each node of hidden layers 504. During training (including forward and backward propagation) of machine learning model 500, the weights for each input are adjusted to establish the relationship between the input data and the mental state output. In some examples, one hidden layer may be incorporated into machine learning model 500, or three or more hidden layers may be incorporated into machine learning model 500, where each layer includes the same or different number of nodes.

The result of each node within hidden layers 504 is applied to the transfer function 505 of output layer 506. The transfer function may be liner or non-linear, depending on the number of layers within machine learning model 500. Example non-linear transfer functions may be a sigmoid function or a rectifier function. The output 507 of the transfer function may be a classification of a particular mental state and/or an index value indicative of mental state.

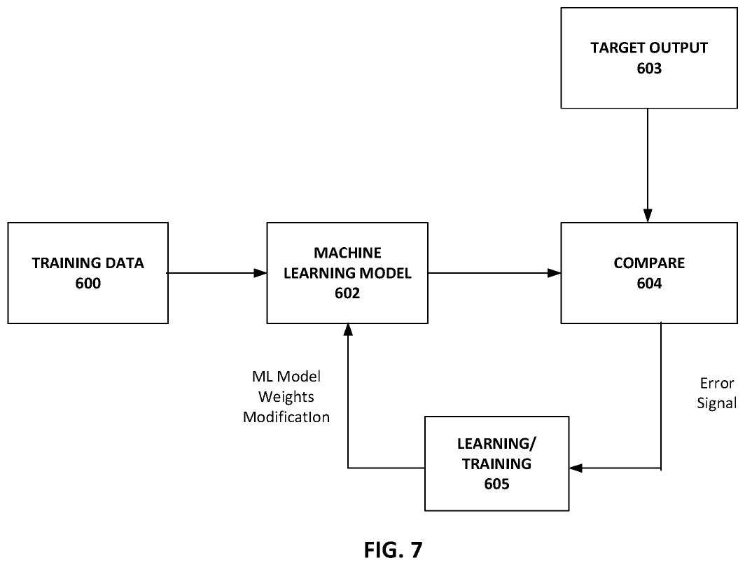

FIG. 7 is a block diagram illustrating training a machine learning model 602, which may be an example of models 84, 196, 250, or 500, being trained using supervised and/or reinforcement learning techniques. The machine learning model 602 may be implemented using any number of models for supervised and/or reinforcement learning, such as but not limited to, an artificial neural network, a decision tree, naïve Bayes network, support vector machine, or k-nearest neighbor model, to name only a few examples. In some examples, one or more of IMD 10, computing device 12 (model configuration 174), and/or HMS 22 (model configuration 234) initially trains the machine learning model 602 based on one or more training sets of features corresponding to different mental states or mental state degrees/likelihoods. In some examples, one or more machine learning models 602 may be developed by identifying training data 600.

Training data 600 may include a set of feature vectors, where each feature in the feature vector represents a value for a particular metric. A training set comprises a set of training instances, each training instance comprising an association between one or more respective signal, parameter, or feature values, and a respective mental state output. One or more experts may annotate the training instances with one or more target outputs, e.g., classifications.

A prediction or classification by the machine learning model 602 may be compared 604 to the target output 603, e.g., which may be based on the labeled classification, and learning/training 605 may include an error signal and/or machine learning model weights modification being sent/applied to the machine learning model 602 based on the comparison to modify/update the machine learning model 602. For example, one or more of IMD 10, computing device 12, and/or HMS 22 may, for each training instance in the training set, modify, based on the respective features and the respective classification of the training instance, the machine learning model 602 to change a score generated by the machine learning model 602 in response to subsequent sets of features applied to the machine learning model 602.

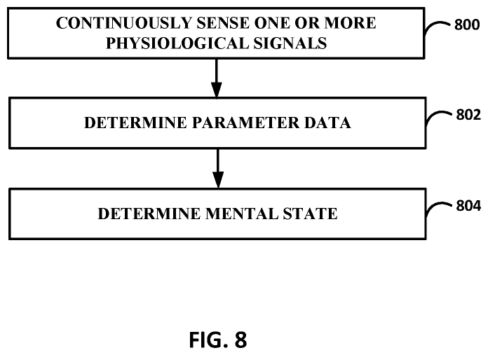

FIG. 8 is a flow diagram illustrating an example operation for determining a mental state of a subject, e.g., patient 4, according to the techniques of this disclosure. According to the example of FIG. 8, one or more implantable monitoring devices, e.g., IMD(s) 10, continuously sense one or more physiological signals of patient 4, as described above (800). In some examples, IMD(s) 10 determine parameter data for one or more parameters based on the physiological signals (802). Processing circuitry of system 2, e.g., processing circuitry 50 of IMD(s) 10, processing circuitry 130 of computing device(s) 12, and/or processing circuitry of a cloud computing system implementing HMS 22, determines a mental state of patient 4 based on the physiological signal(s) and/or parameter data, e.g., by application of the data to a model, such as a machine learning model (804).

Self-reporting of symptoms, including use of questionnaires, are the most common tool to assess a patient's mental state, such as the current state of a mood disorder. However, even when patients are aware of their mental state, they may nevertheless neglect symptoms due to the deepening depression or other worsening mental state. For example, patients may have feelings of inadequacy or shame that cause them to feel they do not need or deserve to be treated well, or they may feel distracted or lack the energy to engage in normal activities, including tending to their mental and physical well-being. Questionnaires suffer the disadvantages of being subjective and point-in-time, and thus may not fully identify the mental state and underlying causes thereof. Patients may not know when to administer pro-re-nata (PRN) treatments, e.g., medication, mental health check, or self-care activities, until symptoms have already worsened, or they may forget or neglect to administer chronic treatment, e.g., daily medication.

A system that determines mental states, including a medical device that continuously monitors physiological signals, may address these shortcomings of self-reporting and infrequent clinician observation. In some examples, such a system includes processing circuitry of a computing device that is configured to determine mental states of a subject based on biomarkers sensed by an IMD, e.g., including any of the physiological signals and/or derived parameters described herein. Such biomarkers may be more objective, reliable, and accurate indicators of a subject's mental state than a subject's subjective self-evaluation alone. In this way, the IMD's ability to sense biomarkers, and the configuration of processing circuity to determine mental states may provide a technological advantage over subjective self-evaluations and/or discontinuous/ad hoc measurements that rely on patient compliance for a cohort group that is already predisposed to struggling with remembering, desiring, or having the energy to fill out questionnaires.

As described above, the physiological signals may include one or more of an ECG, an EEG, heart sounds signal, blood pressure signal, an oxygen saturation signal, a skin conductance signal, a respiration signal, a motion signal (e.g., activity), a posture signal, or a temperature signal. Parameter data that may be determined based on the ECG signal include heart rate, heart rate variability, heart rate during certain diurnal periods, e.g., day or night, and morphology of various waves in the ECG. Heart rate variability and blood pressure, for example, may indicate autonomic function, which may be influenced by mental state, and changes therein may reflect mental state. Parameter data that may be determined based on the EEG signal include morphology and energy levels (e.g., spectral power) in different frequency bands, or bispectral index via bispectrum analysis. Parameter data that may be determined based on the EEG signal may include a ratio or other metric of comparison between energy levels in two or more frequency bands or between brain hemispheres or different brain locations. Power in lower frequency bands may associated with better mental state, and power in higher frequency bands associated with worsening mental state. Parameter data that may be determined based on the HS signal may include electromechanical activation time, strength of heart sounds S1 and S2, heart sound S3 and S4, systolic interval, etc. Acoustic signal might also help sense the patient speech and provide indication when and how the patient interacts with others, etc.

Parameter data that may be determined based on other physiological signals may include maximum, minimum, mean, or variability values for different periods, e.g., minute, hour, or day. Parameter data that may be determined based on a posture signal may include amounts of time spent in one or more postures or number/frequency of posture transitions. Parameter data based on a posture signal is of interest, as it can be indicative of several mental states and disorders. For example, bipolar disorders are characterized by periods of mania and high activity alternating with periods of depression and low activity, while schizophrenia and depression result in characteristic movement patterns or lack of movement.

In some examples, processing circuitry may determine whether patient 4 is asleep and/or quality of sleep based on the physiological signals or parameter data determined from the physiological signals. The processing circuitry may determine the mental state of patient 4 based on the sleep quality and/or patterns. Sleep quality and/or patterns may include variability in sleep duration, sleep onset, efficiency, transitions between sleep phases, rapid eye movement patterns, and number of awakenings during the sleep time.

In some examples, processing circuitry may determine a menstrual cycle state based on the physiological signals or parameter data determined from the physiological signals or from the device's clock. For example, temperature varies slightly with menstrual cycle. In some examples, processing circuitry may apply physiological signals and/or parameter data determined from the physiological signals to a machine learning model to determine the menstrual cycle state and/or predict the next menstruation.

In some examples, processing circuitry may determine a degree of malnutrition based on one or more of an ECG signal, impedance, and/or galvanic skin response. Hydration may decrease with malnutrition. Also, malnutrition may impact heart rate variability, ECG morphology, and/or frequency of arrhythmia. Processing circuitry may determine a state of anorexia or other mental disorders based on a degree of malnutrition.

In some examples, the processing circuitry may determine the mental state of the patient based on physiological parameter data and other data collected computing device(s) 12 and/or IoT device(s) 30. The data may include survey/questionnaire data regarding symptoms, sleep patterns, activities, compliance with treatment/self-care, and diet. Poor diet, including junk food, can be a factor in deepening depression. Poor social contacts and tendency of social withdrawal and isolation are also factors in deepening depression. The data collection via computing device(s) 12 and/or IoT devices may correspond to data collected via the PHQ-9 questionnaire.

In some examples, IoT devices 30 may provide patient parameter data via microphones, cameras, and LIDAR sensors to track activity of patient 4. In some examples, patient parameter data may include usage of computing devices 12 indicative of mental state, such as amount, patterns, times of day, applications used, websites visited, changes in interactions with certain social contacts or other predefined individuals, etc. Location tracking of patient 4 based on computing device 12 location may also be indicative of mental state, e.g., changes in movement or movement to unusual locations.

Parameter data collected and analyzed as described herein may allow identification of onset/worsening of depression or other mental states, as well as improvements in mental states. In some examples, based on the determination of the mental state, (e.g., indicating onset, worsening, or improving), the processing circuitry may provide messages via computing devices 12, 38 to patient 4, the patient's healthcare provider or, potentially, trusted friends or family members who can intervene to help the patient through difficult periods. With respect to the patient, the message may prompt action on the part of the patient, such as PRN dosages of medication or self-care activities, such as diet, exercise, medication, therapy, e.g., using an external neurostimulator, or mediation/relaxation.

In some examples, the analysis may be via a machine learning model as described above. In some examples, the analysis may include comparison of multiple parameter values to respective baselines or thresholds, e.g., comparing current values or short-term averages to long-term averages. In some examples, an indication for monitoring or the model(s) used by system 2 to monitor mental states of patient 4 may be selected based on a condition, cohort, comorbidity, or location of patient 4, such as depression, pregnancy, post-partum status, cancer diagnosis, stroke, heart attack, heart failure, trauma, location in menstrual cycle, cardiac surgery, occupation, geographic location, or weather.

Changes in mental state determined by system 2 could cause HMS 22 and/or computing devices 12 to prompt patient with questions to further clarify the mental state or inform clinicians and other interested users. In addition to family and friends, interested users could include employers of individuals in high stress situations, such as police, fire, emergency responders, or military. In some cases, such users may have a dashboard via HMS 22 and computing devices 38 to assess the mental state of the monitored individual. The dashboard may include a plurality of individuals, e.g., a team, and data indicating determined mental state for each individual, such as an index value, trend over time, and annotations generated by processing circuitry to highlight the relative mental state or risk associated with each individual.

In some examples, in addition to mental state, system 2 may be configured to identify a suicide attempt based on patient parameter data, e.g., changes in physiological parameters or movement associated with drug overdose, slit wrists, carbon monoxide poisoning (e.g., via oxygen saturation), or gun shots. In such examples, IMDs 10, computing devices 12, IoT devices 30, and/or HMS 22 may initiate an alert protocol that will result in rapid communication to EMS or other parties.

In some examples, system 2 may be configured to monitor compliance of patient 4 with a medication regimen. In such examples, IMD(s) 10 may analyze parameter data of patient 4 associated with physiological changes caused by the medication, e.g., within a window around the scheduled time for a dose. In some examples, IMD(s) 10 or other devices of system may be configured with one or more sensors to detect a marker or communication mechanism associated with a pill or medication container.

In some examples, the processing circuitry is configured to receive data indicating a comorbid condition of patient 4 and determine the mental state of the subject based on the data indicating the comorbid condition. For example, processing circuitry may include, as input to a model used to determine mental state, a value indicating the presence and/or degree of one or more comorbid conditions. Example comorbid conditions include heart attack, stroke, cardiac surgery, defibrillation shock, traumatic injury, heart failure, diabetes, post-traumatic stress disorder, post-partum, or cancer. The processing circuitry may receive the data indicating the comorbid condition from EHR 24.

In some examples, system 2 may be configured to determine the state of one or more comorbid conditions, such as heart failure or diabetes, based on physiological signals and parameter data, e.g., collected by IMD(s) 10 and computing device(s) 12. Physiological signals from which processing circuitry may determine a heart failure state of patient 4 may include, for example, an ECG signal, a respiration signal, a motion signal, a subcutaneous impedance signal, a blood pressure signal, or a heart sounds signal. Example techniques for determining a heart failure state based on physiological signals and parameter data determined from physiological signals are described in commonly assigned U.S. Pat. No. 10,542,887 by Sarkar et al., titled “HEART FAILURE MONITORING,” which is incorporated herein by reference in its entirety. In some examples, processing circuitry may use the mental state, e.g., an index value of a mental state, as an input value for the determination of the heart failure state.

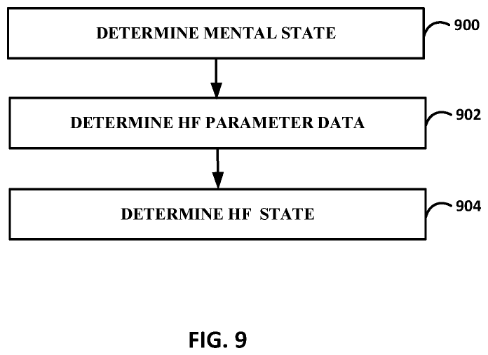

FIG. 9 is a flow diagram illustrating an example operation for determining a heart failure state of a subject, e.g., patient 4, according to the techniques of this disclosure. According to the example of FIG. 9, processing circuitry of system 2, e.g., processing circuitry 50 of IMD(s) 10, processing circuitry 130 of computing device(s) 12, and/or processing circuitry of a cloud computing system implementing HMS 22, determines a mental state of patient 4 based on physiological signal(s) and/or parameter data, e.g., according to the example operation of FIG. 8 (900). The processing circuity also receives/determines parameter data for HF, e.g., based on the physiological signals described above (902). The processing circuitry determines a heart failure state of patient 4 based on the HF parameter data and the mental state (904). The heart failure state may be an index value (e.g., low, medium, or high, or numerical) indicating the risk, likelihood, or probability of a heart failure event, such as worsening, decompensation, or hospitalization.

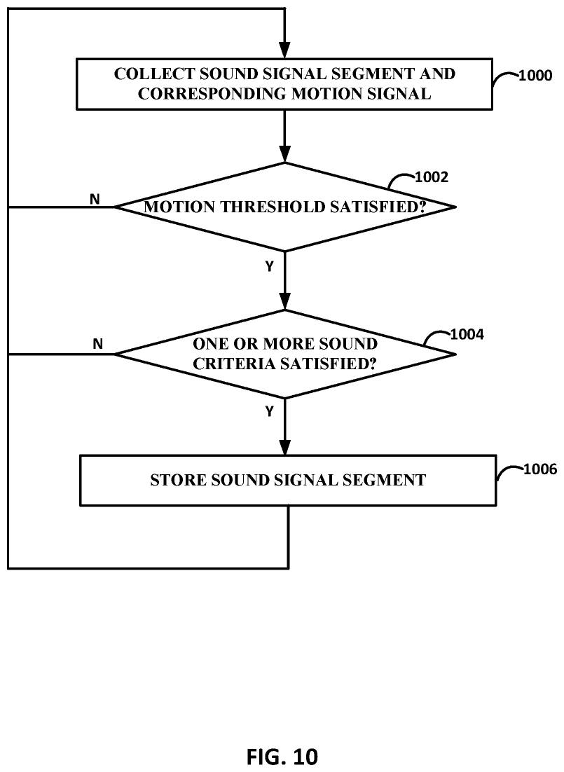

FIG. 10 is a flow diagram illustrating an example operation for identifying sound signal segments suitable for voice characteristic measurement according to the techniques of this disclosure. As described above, IMD 10 may include a sound sensor, e.g., a piezo electric crystal sensor or microphone, within, e.g., attached to, its housing. The sound sensor may be configured to sense the sounds associated with the voice of patient 4. IMD 10 may filter the sound signal to include voice frequencies, e.g., 85-255 Hz, or may filter according to gender to reduce noise, e.g., 85-155 Hz for males and 165-255 Hz for females.

IMD 10 may collect a segment, e.g., between 20 milliseconds and 1 second, such as 50 milliseconds, of the filtered sound signal and a corresponding segment of the motion signal (1000). Processing circuitry 50 of IMD 10 may determine whether the motion signal segment satisfies a motion threshold (1002). For example, processing circuitry 50 may determine activity counts or another metric of the amount of motion during the segment and determine whether the metric is less than the motion threshold. Sound segments with timing corresponding to sub-threshold motion are less likely to have signal noise associated with the motion and can be compared to a baseline signal having a similar signal noise condition.

If the motion signal segment does not satisfy the motion threshold (NO of 1002), processing circuitry 50 collects the next sound and motion signal segments (1000). If the motion signal segment satisfies the motion threshold (YES of 1002), processing circuitry 50 determines whether the sound signal satisfies one or more sound signal criteria (1004). The one or more sound criteria may comprise one or more of an energy criterion or a zero-crossing criterion, e.g., thresholds for each of energy and zero-crossings that must be met or exceeded thereby indicating signal activity associated with the voice of patient 5. If the sound signal segment does not satisfy the one or more sound criteria (NO of 1004), processing circuitry 50 collects the next sound and motion signal segments (1000). If the sound signal segment satisfies the one or more sound signal criteria (YES of 1004), processing circuitry 50 stores the sound signal segment for further analysis of voice characteristics to determine the mental state of patient 4, which may include transmitting the sound signal segment to computing device(s) 12 and/or the cloud computing system implanting HMS 22 (1006).

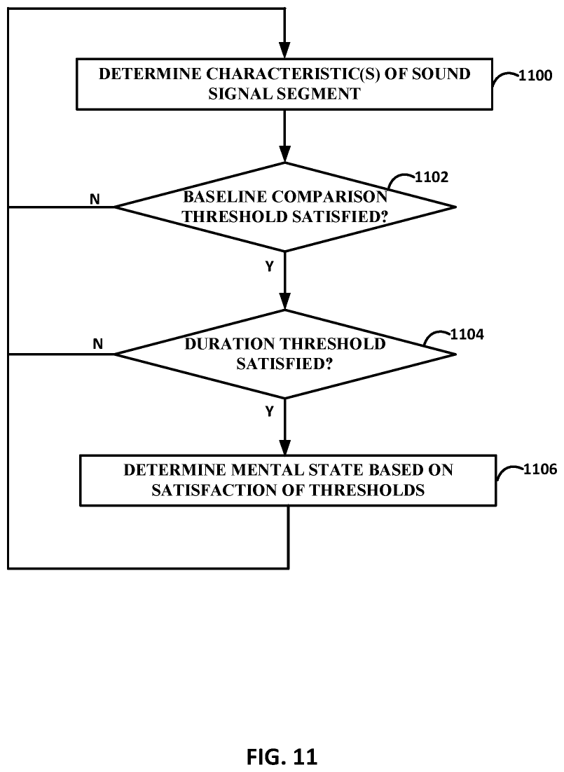

FIG. 11 is a flow diagram illustrating an example operation for determining a mental state of a subject, e.g., patient 4, based on changes in subject voice characteristics according to the techniques of this disclosure. Voice characteristics change based on mental state, such as during depression. According to the example of FIG. 11, processing circuitry of system 2, e.g., processing circuitry 50 of IMD(s) 10, processing circuitry 130 of computing device(s) 12, and/or processing circuitry of a cloud computing system implementing HMS 22, determines parameter values for each of one or more voice characteristics based on the sound signal for each of a plurality of sound signal segments (1100). Voice characteristics include pitch, volume, pause, and rate. In some examples, the processing circuitry may monitor patient 4 for objective signs of depression including voice characteristics.

The processing circuitry compares the voice characteristic values to baseline values of the voice characteristics. In some examples, patient 4 is prompted, e.g., by computing device(s) 12, to be relatively still and speak, e.g., a certain series of words at a requested volume level, to provide sound signal segments for determining the baseline voice characteristic values. The computing device may communicate with IMD 10 so that IMD 10 records the sound signal segments for generation of the baseline characteristic values.

The processing circuitry determines whether the comparison of the characteristic values for the segment or a period comprising a plurality of segments with the baseline characteristic values satisfies a comparison threshold, e.g., whether differences relative to the baseline exceed a threshold (1102). If the comparison threshold is not satisfied (NO of 1102), the processing circuitry determines the characteristic values for another segment (1100). If the comparison metric is satisfied (YES of 1102), the processing circuitry determines whether a duration threshold is satisfied, e.g., based on whether a plurality of consecutive periods or signal segments satisfied the comparison threshold (1104). If the duration threshold is not satisfied (NO of 1104), the processing circuitry determines the characteristic values for another segment (1100). If the duration threshold is satisfied (YES of 1104), the processing circuitry determines the mental state of patient 4 (1106).

In some examples, the duration threshold may be whether the sound signal of y consecutive periods or segments, or x of y consecutive periods or segments, satisfied the comparison threshold for at least m of n characteristics. In some examples, the processing circuitry may trend voice episode characteristics, determine periodic min/max/median for each of the voice characteristics, determine percent or other amount of change from the baseline. In an example, if y continuous days (e.g., 7) have m of n voice characteristics over a threshold percent change relative to the baseline, processing circuitry may classify patient 4 as depressed, and trigger messages to one or more users as described above. The patient may receive therapy to treat their depression, which may reduce the risk of an adverse cardiac event. In some examples, processing circuitry may apply values indicating whether thresholds for one or more voice characteristics and/or the duration threshold were satisfied, or the voice characteristic values or sound signal segments themselves, as inputs to a model, e.g., with other signals and/or parameter data as described herein, to determine a mental state of patient 4.

In some examples, the IMD or other device that collects for the voice segments, or the processing circuitry that analyzes the voice segments, may analyze characteristics of the segments to determine whether the sound signal is the voice of the subject rather than another person in the vicinity of the subject. The voice of each individual may have a distinct signature represented in the characteristics of the sound signal. Processing circuitry may compare the characteristics of each sound segment to a baseline for the subject to confirm it is of the subject. If the sound segment is not confirmed to be of the subject, the processing circuitry may not include it in the analysis for the mental state of the subject.

Another example of a mental state of patient 4 that may be determined according to the techniques described herein is confusion. Identifying a decline in the health of a patient (e.g., an elderly person) may help them receive earlier treatment for the cause of the decline. An increase in confusion may indicate onset or worsening of a medical condition. Confusion may be due to, for example, dehydration, dementia, and other health changes.

An increase in confusion may lead to a significant change in movement patterns (e.g., wandering around a patient's living residence, such as a house) compared to a baseline. In accordance with techniques disclosed here, a medical device system may monitor the movement patterns of a patient to assess a level of confusion. For example, one or more IMDS 10, computing devices 12 and/or IoT devices 30 may collect data indicting locations of patient 4 and corresponding times, and processing circuitry of the system may determine a confusion state of patient 4 based on the data. Example sensors include GPS, cameras, magnetometers, gyroscopes, etc.

The system may have an initialization period (e.g., 7 days) to determine a baseline for the movement patterns. Sensors may continuously monitor the patient (e.g., 24/7). Deviations from the baseline may indicate confusion, onset of a medical condition (e.g., dementia), and/or worsening of a diagnosed medical condition. Processing circuitry may identify characteristics of the movement based on the data, such as pauses or changes in direction. Pauses and changes in direction may indicate confusion (e.g., the patient is trying to remember something).

Responsive to the processing circuitry determining a confusion state, onset of a medical condition (e.g., dementia), and/or worsening of a diagnosed medical condition, computing devices 12 and/or 38, such as notifying the patient of the system's determination, recommending an intervention (e.g., drinking water), notifying a caretaker (e.g., the patient's primary physician or caregiver), etc. Computing devices 12, 38 and/or IoT devices 30 may also receive user input (e.g., audio input). The external device may query the patient regarding patient history (e.g., diagnosed mental conditions), daily activities, etc. To reduce false-positive determinations, the system may ask the patient questions about the deviations from the baseline (e.g., were you doing anything unusual at 12:00 PM today?). The system may perform an action described above at least partly based on the input from the patient.

In an example, a system comprises a location unit configured to continuously receive location data associated with a patient, an implantable medical device comprising a sensor configured to measure a parameter indicative of a hydration status of a patient (e.g., electrodes 56 configured to measure tissue impedance), and one or more computing devices comprising communication circuitry configured to receive data indicative of the hydration status of the patient from the implantable medical device. The system further comprises processing circuitry configured to: determine, based on the location data, a first set of locations of the patient during an initialization period; determine, based on the location data, a second set of locations of the patient during an operation period, wherein the operation period is after the initialization period; determine a deviation between the second set of locations and the first set of locations; determine whether the deviation satisfies a deviation threshold; determine whether the hydration status satisfies a dehydration condition; responsive to determining that the deviation satisfies the deviation threshold and determining that the hydration status satisfies the dehydration condition, output a notification indicating a change in a health status of the patient attributable to the patient being dehydrated; and responsive to determining that the deviation satisfies the deviation threshold and determining that the hydration status does not satisfy the dehydration condition, output a notification indicating a change in a health status of the patient that is not attributable to dehydration.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

Example 1. A system comprising: one or more implantable monitoring devices configured to continuously sense a plurality of physiological signals of a subject and collect parameter data of the subject based on the sensed physiological signals, wherein at least one implantable monitoring device of the one or more implantable monitoring devices comprises a housing configured for subcutaneous implantation in the subject and a plurality of electrodes positioned on the housing, wherein the at least one implantable monitoring device is configured to continuously sense at least one physiological signal of the plurality of physiological signals via the plurality of electrodes; and processing circuitry of one or more of: the at least one implantable monitoring device; one or more computing devices configured to wirelessly communicate with the one or more implantable monitoring devices; or a cloud computing system configured to communicate with at least one of the one or more implantable monitoring devices or the one or more computing devices, the processing circuitry configured to determine a mental state of the subject based on at least one of the sensed physiological signals or the parameter data.

Example 2. The system of example 1, wherein the at least one implantable monitoring device comprises an insertable cardiac monitor configured to sense an electrocardiogram of the subject via the plurality of electrodes.

Example 3. The system of example 1, wherein the housing of the at least one implantable monitoring device is configured for subcutaneous implantation on a head or neck of the subject, and the at least one implantable monitoring device is configured to sense an electroencephalogram (EEG) of the subject via the plurality of electrodes.

Example 4. The system of example 2, wherein the at least one implantable monitoring device comprises a first implantable monitoring device comprising a first housing and a first plurality of electrodes, wherein the one or more implantable monitoring devices comprise a second implantable monitoring device comprising a second housing configured for subcutaneous implantation on a head or neck of the subject and a second plurality of electrodes on the housing, wherein the second implantable monitoring device is configured to sense an electroencephalogram (EEG) of the subject via the second plurality of electrodes.

Example 5. The system of example 3 or 4, wherein the processing circuitry configured to determine the mental state of the subject based a morphology of the EEG.

Example 6. The system of any one or more of examples 3 to 5, wherein the processing circuitry is configured to determine the mental state of the subject based on a respective energy level in one or more frequency bands or sensing locations of the EEG.

Example 7. The system of example 6, wherein the processing circuitry is configured to determine the mental state of the subject based on a ratio between a first energy level in a first frequency band or sensing location of the EEG and a second energy level in a second frequency band or sensing location of the EEG.

Example 8. The system of any one or more of examples 1 to 7, wherein the at least one implantable monitoring device comprises an accelerometer and a plurality of physiological signals comprise a signal from the accelerometer indicative of at least one of motion or posture of the subject.

Example 9. The system of any one or more of examples 1 to 8, wherein the sensed physiological signals comprise one or more of a blood pressure signal, an oxygen saturation signal, a skin conductance signal, a respiration signal, or a temperature signal.

Example 10. The system of example 9, wherein the parameter data comprises a variability of one or more of the blood pressure signal, the oxygen saturation signal, the skin conductance signal, the respiration signal, or the temperature signal.

Example 11. The system of any one or more of examples 1 to 10, wherein the processing circuitry is configured to: determine at least one of sleep quality or sleep patterns of the subject based on one or more of the physiological signals or parameter data; and determine the mental state of the subject based on the at least one of the sleep quality or sleep patterns.

Example 12. The system of any one or more of examples 1 to 11, wherein the one or more computing devices comprise a computing of the subject comprising at least one of a smartwatch, smartphone, or Internet of Things device.

Example 13. The system of example 12, wherein the computing device of the subject is configured to determine locations of the subject over time, and the processing circuitry is configured to determine the mental state of the subject based on the determined locations over time.

Example 14. The system of example 13, wherein the processing circuitry is configured to determine the mental state of the subject based on at least one of an amount or pattern of movement determined based on the locations over time.

Example 15. The system of any one or more of examples 12 to 14, wherein the computing device of the subject is configured to monitor interactions of the subject with the computing device, and the processing circuitry is configured to determine the mental state of the subject based on the interactions.

Example 16. The system of example 15, wherein the processing circuitry is configured to determine the mental state of the subject based on at least one of an amount or pattern of the interactions.

Example 17. The system of 15 or 16, wherein the interactions comprise interactions with one or more social media accounts of the subject.

Example 18. The system of any one or more of examples 15 to 17, wherein the interactions comprise interactions with one or more predefined individuals using the computing device.

Example 19. The system of any one or more of examples 1 to 18, further comprising a sensor configured to sense a voice of the subject, wherein the processing circuitry is configured to determine the mental state of the subject based on values of one or more characteristics of the voice of the subject.

Example 20. The system of example 19, wherein the one or more characteristics comprise one or more of speech rate, speech rate variability, pitch, pitch variability, length of pauses, frequency of pauses, or pattern of pauses.

Example 21. The system of example 19 or 20, wherein to determine the mental state of the subject, the processing circuitry is configured to compare the values of the one or more characteristics to baseline values of the one or more characteristics.

Example 22. The system of any one or more of examples 19 to 21, wherein the values of the one or more characteristics comprise one or more of maximum value, minimum values, or median values of the one or more characteristics during a time period.

Example 23. The system of example 22, wherein a plurality of consecutive time periods comprise the time period, and to determine the mental state of the subject the processing circuitry is configured to: determine whether the comparison of the values of the one or more characteristics to the baseline satisfies a comparison threshold; and determine whether the comparison threshold is satisfied for a threshold amount of the plurality of consecutive periods.

Example 24. The system of any one or more of examples 19 to 23, wherein the sensor configured to sense the voice of the subject is located within the housing of the at least one implantable monitoring device.

Example 25. The system of any one or more of examples 21 to 23, example 12, and example 24, wherein the computing device of the subject is configured to prompt the subject to provide a voice sample, and the processing circuitry is configured to determine the baseline values of the one or more characteristics from the voice sample.

Example 26. The system of any one or more of examples 19 to 23 and example 24 or 25, wherein implantable monitoring device further comprises an accelerometer and is configured to: determine that a motion signal from the accelerometer satisfies a motion threshold and a sound signal from the sound sensor satisfies one or more sound criteria; and store the sound signal for determination of the one or more characteristics of the voice of the subject based on the determination.

Example 27. The system of example 26, wherein the one or more sound criteria comprise one or more of an energy criterion or a zero-crossing criterion.

Example 28. The system of any one or more of examples 19 to 23, wherein the one or more computing devices comprise a computing device of the subject comprising a smartwatch, smartphone, or Internet of Things device of the subject, and the sensor configured to sense the voice of the subject is located within the computing device of the subject.

Example 29. The system of any one or more of examples 1 to 28, wherein the mental state comprises one or more of a mood disorder state, a fatigue state, or an alertness state.

Example 30. The system of example 29, wherein the mood disorder state comprises a depression state, an anxiety state, a schizophrenia state, a bipolar disorder state, post-traumatic stress disorder state, or a menstrually-related mood disorder state.

Example 31. The system of example 30, wherein the menstrually-related mood disorder state comprises a pre-menstrual syndrome state, a pre-menstrual dysphoric disorder state, a perimenopausal depression state, or a post-partum depression state.

Example 32. The system of any one or more of examples 1 to 31, wherein the processing circuitry is configured to: determine a menstruation state of the subject based on at least one of the sensed physiological signals or the parameter data; and determine the mental state of the subject based on the menstruation state.

Example 33. The system of any one or more of examples 1 to 32, wherein the processing circuitry is configured to: receive data indicating a comorbid condition of the subject; and determine the mental state of the subject based on the data indicating the comorbid condition.

Example 34. The system of example 33, wherein the comorbid condition comprises heart attack, stroke, cardiac surgery, defibrillation shock, traumatic injury, heart failure, post-traumatic stress disorder, post-partum, or cancer.

Example 35. The system of any one or more of examples 1 to 34, wherein the plurality of physiological signals comprises a first plurality of physiological signals and the parameter data comprises first parameter data, and the one or more implantable monitoring devices are configured to continuously sense a second plurality of physiological signals of the subject and collect second parameter data of the subject based on the second plurality of physiological signals, wherein the processing circuitry is configured to determine a heart failure state of the subject based on at least one of the second physiological signals or the second parameter data.

Example 36. The system of example 35, wherein the second plurality of physiological signals comprise one or more of an electrocardiogram signal, a respiration signal, a motion signal, a subcutaneous impedance signal, a blood pressure signal, or a heart sounds signal.

Example 37. The system of example 35 or 36, wherein the processing circuitry is configured to determine the heart failure state based on the mental state.

Example 38. The system of any one or more of examples 1 to 34, wherein the plurality of physiological signals comprises a first plurality of physiological signals and the parameter data comprises first parameter data, the system further comprising one or more wearable monitoring devices that are configured to sense at least one second physiological signal of the subject and collect second parameter data of the subject based on the second physiological signal, wherein the processing circuitry is configured to determine the mental state of the subject based on at least one of the second physiological signal or the second parameter data.

Example 39. The system of any one or more of examples 1 to 38, wherein at least one of the one or more computing device or the cloud computing system is configured to prompt a user for input, and the processing circuitry is configured to determine the mental state of the subject based on the input.

Example 40. The system of example 39, wherein the user comprises the subject, a caregiver of the subject, a clinician of the subject, a family member of the subject, or a friend of the subject.

Example 41. The system of example 39 or 40, wherein the input relates to one or more of menstruation of the subject, diet of the subject, activity of the subject, or sleep of the subject.

Example 42. The system of any one or more of examples 1 to 41, where to determine the mental state of the subject based on the at least one of the sensed physiological signals or the parameter data, the processing circuitry is configured to apply the at least one of the sensed physiological signals or the parameter data to a machine learning model, the machine learning model trained to generate an output indicating the mental state using a training set comprising a plurality of examples of at least one of sensed physiological signals or parameter data labeled with a respective one of a plurality of mental states.

Example 43. The system of any one or more of examples 1 to 42, wherein at least one of the one or more computing devices or the cloud computing system is configured to present an output to a user based on determined mental state.

Example 44. The system of example 43, wherein the user comprises the subject, a caregiver of the subject, a clinician of the subject, a family member of the subject, or a friend of the subject.

Example 45. The system of example 43 or 44, wherein the output comprises the mental state.

Example 46. The system of example 45, wherein the output comprises a plurality of mental states of the subject determined by the processing circuitry over time.

Example 47. The system of example 45 or 46, wherein the output comprises a dashboard comprising the mental state determined for a plurality of subjects.

Example 48. The system of any one or more of examples 43 to 47, wherein the mental state comprises an index value.

Example 49. The system of any one or more of examples 43 to 48, wherein the processing circuitry is configured to: determine that the mental state satisfies at least one mental state criterion; and determine the output based on satisfaction of the at least one mental state criterion.

Example 50. The system of example 49, wherein the output comprises at least one of an alert, or a recommendation of an activity or therapy for the subject, or an instruction for the subject.

Example 51. The system of any one or more of examples 1 to 50, wherein the processing circuitry is further configured to detect a suicide attempt of the subject based on the at least one of the sensed physiological signals or the parameter data.

Example 52. The system of any one or more of examples 1 to 51, wherein the processing circuitry is configured to: detect crying episodes based on at least one of a respiration or motion signal;
determine at least one of a frequency or duration of the crying episodes; and determine the mental state of the subject based on the at least one of the frequency or duration of the crying episodes.

Example 53. A medical system comprising: an insertable cardiac monitor comprising: a housing configured for subcutaneous implantation in a subject, the housing having a length between 40 millimeters (mm) and 60 mm between a first end and a second end, a width less than the length, and a depth less than the width; a first electrode at or proximate to the first end; a second electrode at or proximate to the second end; sensing circuitry within the housing, the sensing circuitry configured to continuously sense a plurality of physiological signals including an at least an electrocardiogram of the subject via the first electrode and the second of electrode; a memory within the housing; and first processing circuitry within the housing, the first processing circuitry configured to collect parameter data of the subject based on the sensed physiological signals; and one or more computing devices in communication with the insertable cardiac monitor, the one or more computing devices comprising second processing circuitry configured to determine a mental state of the subject based on at least one of the sensed physiological signals or the parameter data.

Example 54. A method for operating a system comprising one or more implantable monitoring devices to determine a mental state of a subject, wherein at least one implantable monitoring device of the one or more implantable monitoring devices comprises a housing configured for subcutaneous implantation in the subject and a plurality of electrodes positioned on the housing, wherein the at least one implantable monitoring device is configured to continuously sense at least one physiological signal of the plurality of physiological signals via the plurality of electrodes, the method comprising: continuously sensing, by the one or more implantable monitoring devices, a plurality of physiological signals of the subject; collecting, by the one or more implantable monitoring devices, parameter data of the subject based on the sensed physiological signals; and determining, by processing circuitry, a mental state of the subject based on at least one of the sensed physiological signals or the parameter data, wherein the processing circuitry comprises processing circuitry of one or more of: the at least one implantable monitoring device; one or more computing devices configured to wirelessly communicate with the one or more implantable monitoring devices; or a cloud computing system configured to communicate with at least one of the one or more implantable monitoring devices or the one or more computing devices.

Example 55. The method of example 54, wherein the at least one implantable monitoring device comprises an insertable cardiac monitor and continuously sensing a plurality of physiological signals comprises continuously sensing an electrocardiogram of the subject via the plurality of electrodes.

Example 56. The method of example 54, wherein the housing of the at least one implantable monitoring device is configured for subcutaneous implantation on a head or neck of the subject, and continuously sensing a plurality of physiological signals comprises continuously sensing an electroencephalogram (EEG) of the subject via the plurality of electrodes.

Example 57. The method of example 55, wherein the at least one implantable monitoring device comprises a first implantable monitoring device comprising a first housing and a first plurality of electrodes, wherein the one or more implantable monitoring devices comprise a second implantable monitoring device comprising a second housing configured for subcutaneous implantation on a head or neck of the subject and a second plurality of electrodes on the housing, wherein continuously sensing a plurality of physiological signals comprises continuously sensing an electroencephalogram (EEG) of the subject via the second plurality of electrodes.

Example 58. The method of example 56 or 57, wherein determining the mental state comprises determining the mental state of the subject based a morphology of the EEG.

Example 59. The method of any one or more of examples 56 to 58, wherein determining the mental state comprises determining the mental state the mental state of the subject based on a respective energy level in one or more frequency bands or sensing locations of the EEG.

Example 60. The method of example 59, wherein determining the mental state comprises determining the mental state the mental state of the subject based on a ratio between a first energy level in a first frequency band or sensing location of the EEG and a second energy level in a second frequency band or sensing location of the EEG.

Example 61. The method of any one or more of examples 54 to 60, wherein the at least one implantable monitoring device comprises an accelerometer and plurality of physiological signals comprise a signal from the accelerometer indicative of at least one of motion or posture of the subject.

Example 62. The method of any one or more of examples 54 to 61, wherein the sensed physiological signals comprise one or more of a blood pressure signal, an oxygen saturation signal, a skin conductance signal, a respiration signal, or a temperature signal.

Example 63. The method of example 62, wherein the parameter data comprises a variability of one or more of the blood pressure signal, the oxygen saturation signal, the skin conductance signal, the respiration signal, or the temperature signal.

Example 64. The method of any one or more of examples 54 to 63, further comprising: determining at least one of sleep quality or sleep patterns of the subject based on one or more of the physiological signals or parameter data; and determining the mental state of the subject based on the at least one of the sleep quality or sleep patterns.

Example 65. The method of any one or more of examples 54 to 64, wherein the one or more computing devices comprise a computing of the subject comprising at least one of a smartwatch, smartphone, or Internet of Things device.

Example 66. The method of example 65, further comprising determining locations of the subject over time using the computing device, and wherein determining the mental state comprises determining the mental state of the subject based on the determined locations over time.

Example 67. The method of example 66, wherein determining the mental state comprises determining the mental state of the subject based on at least one of an amount or pattern of movement determined based on the locations over time.

Example 68. The method of any one or more of examples 65 to 67, further comprising monitoring, by the computing device, interactions of the subject with the computing device, and determining the mental state comprises determining the mental state of the subject based on the interactions.

Example 69. The method of example 68, wherein determining the mental state comprises determining the mental state of the subject based on at least one of an amount or pattern of the interactions.

Example 70. The method of 68 or 69, wherein the interactions comprise interactions with one or more social media accounts of the subject.

Example 71. The method of any one or more of examples 68 to 70, wherein the interactions comprise interactions with one or more predefined individuals using the computing device.

Example 72. The method of any one or more of examples 54 to 71, further comprising sensing a voice of the subject with a sensor, wherein determining the mental state comprises determining the mental state of the subject based on values of one or more characteristics of the voice of the subject.

Example 73. The method of example 72, wherein the one or more characteristics comprise one or more of speech rate, speech rate variability, pitch, pitch variability, length of pauses, frequency of pauses, or pattern of pauses.

Example 74. The method of example 72 or 73, wherein determining the mental state comprises comparing the values of the one or more characteristics to baseline values of the one or more characteristics.

Example 75. The method of any one or more of examples 72 to 74, wherein the values of the one or more characteristics comprise one or more of maximum value, minimum values, or median values of the one or more characteristics during a time period.

Example 76. The method of example 75, wherein a plurality of consecutive time periods comprise the time period, and determining the mental state comprises: determining whether the comparison of the values of the one or more characteristics to the baseline satisfies a comparison threshold; and determining whether the comparison threshold is satisfied for a threshold amount of the plurality of consecutive periods.

Example 77. The method of any one or more of examples 72 to 76, wherein the sensor configured to sense the voice of the subject is located within the housing of the at least one implantable monitoring device.

Example 78. The method of any one or more of examples 72 to 76, example 65, and example 77, further comprising prompting the subject to provide a voice sample with the computing device, wherein determining the baseline comprises determining the baseline values of the one or more characteristics from the voice sample.

Example 79. The method of any one or more of examples 72 to 76 and example 77 or 78, wherein implantable monitoring device further comprises an accelerometer and the method further comprises: determining that a motion signal from the accelerometer satisfies a motion threshold and a sound signal from the sound sensor satisfies one or more sound criteria; and storing the sound signal for determination of the one or more characteristics of the voice of the subject based on the determination.

Example 80. The method of example 79, wherein the one or more sound criteria comprise one or more of an energy criterion or a zero-crossing criterion.

Example 81. The method of any one or more of examples 72 to 76, wherein the one or more computing devices comprise a computing device of the subject comprising a smartwatch, smartphone, or Internet of Things device of the subject, and the sensor configured to sense the voice of the subject is located within the computing device of the subject.

Example 82. The method of any one or more of examples 54 to 81, wherein the mental state comprises one or more of a mood disorder state, a fatigue state, or an alertness state.

Example 83. The method of example 82, wherein the mood disorder state comprises a depression state, an anxiety state, a schizophrenia state, a bipolar disorder state, post-traumatic stress disorder state, or a menstrually-related mood disorder state.

Example 84. The method of example 83, wherein the menstrually-related mood disorder state comprises a pre-menstrual syndrome state, a pre-menstrual dysphoric disorder state, a perimenopausal depression state, or a post-partum depression state.

Example 85. The method of any one or more of examples 54 to 84, further comprising: determining a menstruation state of the subject based on at least one of the sensed physiological signals or the parameter data; and determining the mental state of the subject based on the menstruation state.

Example 86. The method of any one or more of examples 54 to 85, further comprising: receiving data indicating a comorbid condition of the subject; and determining the mental state of the subject based on the data indicating the comorbid condition.

Example 87. The method of example 86, wherein the comorbid condition comprises heart attack, stroke, cardiac surgery, defibrillation shock, traumatic injury, heart failure, post-traumatic stress disorder, post-partum, or cancer.

Example 88. The method of any one or more of examples 54 to 87, wherein the plurality of physiological signals comprises a first plurality of physiological signals and the parameter data comprises first parameter data, the method further comprising, by the one or more implantable monitoring devices, continuously sensing a second plurality of physiological signals of the subject and collecting second parameter data of the subject based on the second plurality of physiological signals, the method further comprising determining, by the processing circuitry, a heart failure state of the subject based on at least one of the second physiological signals or the second parameter data.

Example 89. The method of example 88, wherein the second plurality of physiological signals comprise one or more of an electrocardiogram signal, a respiration signal, a motion signal, a subcutaneous impedance signal, a blood pressure signal, or a heart sounds signal.

Example 90. The method of example 88 or 89, wherein determining the heart failure state comprises determining the heart failure state based on the mental state.

Example 91. The method of any one or more of examples 54 to 87, wherein the plurality of physiological signals comprises a first plurality of physiological signals and the parameter data comprises first parameter data, the system further comprising one or more wearable monitoring devices that are configured to sense at least one second physiological signal of the subject and collect second parameter data of the subject based on the second physiological signal, wherein determining the mental state comprises determining the mental state of the subject based on at least one of the second physiological signal or the second parameter data.

Example 92. The method of any one or more of examples 54 to 91, further comprising prompting a user for input via at least one of the one or more computing device or the cloud computing system, and determining the mental state comprises determining the mental state of the subject based on the input.

Example 93. The method of example 92, wherein the user comprises the subject, a caregiver of the subject, a clinician of the subject, a family member of the subject, or a friend of the subject.

Example 94. The method of example 92 or 93, wherein the input relates to one or more of menstruation of the subject, diet of the subject, activity of the subject, or sleep of the subject.

Example 95. The method of any one or more of examples 54 to 94, wherein determining the mental state of the subject based on the at least one of the sensed physiological signals or the parameter data comprises applying the at least one of the sensed physiological signals or the parameter data to a machine learning model, the machine learning model trained to generate an output indicating the mental state using a training set comprising a plurality of examples of at least one of sensed physiological signals or parameter data labeled with a respective one of a plurality of mental states.

Example 96. The method of any one or more of examples 54 to 95, further comprising presenting an output to a user based on determined mental state via at least one of the one or more computing devices or the cloud computing system.

Example 97. The method of example 96, wherein the user comprises the subject, a caregiver of the subject, a clinician of the subject, a family member of the subject, or a friend of the subject.

Example 98. The method of example 96 or 97, wherein the output comprises the mental state.

Example 99. The method of example 98, wherein the output comprises a plurality of mental states of the subject determined by the processing circuitry over time.

Example 100. The method of example 98 or 99, wherein the output comprises a dashboard comprising the mental state determined for a plurality of subjects.

Example 101. The method of any one or more of examples 95 to 100, wherein the mental state comprises an index value.

Example 102. The method of any one or more of examples 95 to 101, further comprising: determining that the mental state satisfies at least one mental state criterion; and determining the output based on satisfaction of the at least one mental state criterion.

Example 103. The method of example 102, wherein the output comprises at least one of an alert, or a recommendation of an activity or therapy for the subject, or an instruction for the subject.

Example 104. The method of any one or more of examples 54 to 103, further comprising detecting a suicide attempt of the subject based on the at least one of the sensed physiological signals or the parameter data.

Example 105. The method of any one or more of examples 54 to 104, further comprising, by the processing circuitry: detecting crying episodes based on at least one of a respiration or motion signal; determining at least one of a frequency or duration of the crying episodes; and determining the mental state of the subject based on the at least one of the frequency or duration of the crying episodes.

Example 106. A non-transitory computer-readable storage medium comprising program instructions that, when executed by processing circuitry of a medical system, cause the processing circuitry to: continuously sense, via one or more implantable monitoring devices, a plurality of physiological signals of the subject; cause the one or more implantable monitoring devices to collect parameter data of the subject based on the sensed physiological signals; and determine a mental state of the subject based on at least one of the sensed physiological signals or the parameter data.

Various examples have been described. These and other examples are within the scope of the following claims.

1. A system comprising:
one or more implantable monitoring devices configured to continuously sense a plurality of physiological signals of a subject and collect parameter data of the subject based on the sensed physiological signals, wherein at least one implantable monitoring device of the one or more implantable monitoring devices comprises a housing configured for subcutaneous implantation in the subject and a plurality of electrodes positioned on the housing, wherein the at least one implantable monitoring device is configured to continuously sense at least one physiological signal of the plurality of physiological signals via the plurality of electrodes; and
processing circuitry of one or more of:
the at least one implantable monitoring device;
one or more computing devices configured to wirelessly communicate with the one or more implantable monitoring devices; or
a cloud computing system configured to communicate with at least one of the one or more implantable monitoring devices or the one or more computing devices,
the processing circuitry configured to determine a mental state of the subject based on at least one of the sensed physiological signals or the parameter data.

2. The system of claim 1, wherein the at least one implantable monitoring device comprises an insertable cardiac monitor configured to sense an electrocardiogram of the subject via the plurality of electrodes.

3. The system of claim 1, wherein the housing of the at least one implantable monitoring device is configured for subcutaneous implantation on a head or neck of the subject, and the at least one implantable monitoring device is configured to sense an electroencephalogram (EEG) of the subject via the plurality of electrodes.

4. The system of claim 3, wherein the processing circuitry configured to determine the mental state of the subject based on a morphology of the EEG, the morphology of the EEG comprising a respective energy level in one or more frequency bands or sensing locations of the EEG.

5. The system of claim 1, wherein the at least one implantable monitoring device comprises an accelerometer and the plurality of physiological signals comprise a signal from the accelerometer indicative of at least one of motion or posture of the subject, wherein the sensed physiological signals further comprise one or more of a blood pressure signal, and oxygen saturation signal, a skin conductance signal, a respiration signal, a chemical sensor signal, or a temperature signal.

6. The system of claim 1, wherein the processing circuitry is configured to:
determine at least one of sleep quality or sleep patterns of the subject based on one or more of the physiological signals or parameter data; and
determine the mental state of the subject based on the at least one of the sleep quality or sleep patterns.

7. The system of claim 1, wherein the one or more computing devices comprise a computing device of the subject configured to determine locations of the subject over time, and the processing circuitry is configured to determine the mental state of the subject based on the determined locations over time.

8. The system of claim 7, wherein the computing device of the subject is configured to monitor interactions of the subject with the computing device, and the processing circuitry is configured to determine the mental state of the subject based on the interactions, wherein the interactions comprise interactions with one of more social media accounts of the subject.

9. The system of claim 1, further comprising a sensor configured to sense a voice of the subject, the sensor being located within the housing of the at least one implantable monitoring device, wherein the processing circuitry is configured to determine the mental state of the subject based on values of one or more characteristics of the voice of the subject, wherein the characteristics comprise one or more of speech rate, speech rate variability, pitch, pitch variability, length of pauses, frequency of pauses, or pattern of pauses.

10. The system of claim 9, wherein the implantable monitoring device further comprises an accelerometer and is configured to:
determine that a motion signal from the accelerometer satisfies a motion threshold and a sound signal from the sound sensor satisfies one or more sound criteria, wherein the one or more sound criteria comprise one or more of an energy criterion or a zero-crossing criterion; and
store the sound signal for determination of the one or more characteristics of the voice of the subject based on the determination.

11. The system of claim 1, wherein the processing circuitry is configured to:
receive data indicating a comorbid condition of the subject; and
determine the mental state of the subject based on the data indicating the comorbid condition, wherein the comorbid condition comprises heart attack, stroke, cardiac surgery, defibrillation shock, traumatic injury, heart failure, post-traumatic stress disorder, post-partum, or cancer.

12. The system of claim 1, wherein the plurality of physiological signals comprises a first plurality of physiological signals and the parameter data comprises first parameter data, and the one or more implantable monitoring devices are configured to continuously sense a second plurality of physiological signals, comprising one or more of an electrocardiogram signal, a respiration signal, a motion signal, a subcutaneous impedance signal, a blood pressure signal, or a heart sounds signal, of the subject and collect second parameter data of the subject based on the second plurality of physiological signals, wherein the processing circuitry is configured to determine a heart failure state of the subject based on at least one of the second physiological signals or the second parameter data.

13. The system of claim 1, wherein to determine the mental state of the subject based on the at least one of the sensed physiological signals or the parameter data, the processing circuitry is configured to apply the at least one of the sensed physiological signals or the parameter data to a machine learning model, the machine learning model trained to generate an output indicating the mental state using a training set comprising a plurality of examples of at least one of sensed physiological signals or parameter data labeled with a respective one of a plurality of mental states.

14. The system of claim 1, wherein the processing circuitry is configured to:
determine that the mental state satisfies at least one mental state criterion; and
determine the output based on satisfaction of the at least one mental state criterion, wherein the output comprises at least one of an alert, a recommendation of an activity or therapy for the subject, or an instruction for the subject.

15. A medical system comprising:
an insertable cardiac monitor comprising:
a housing configured for subcutaneous implantation in a subject, the housing having a length between 40 millimeters (mm) and 60 mm between a first end and a second end, a width less than the length, and a depth less than the width;
a first electrode at or proximate to the first end;
a second electrode at or proximate to the second end;
sensing circuitry within the housing, the sensing circuitry configured to continuously sense a plurality of physiological signals including an at least an electrocardiogram of the subject via the first electrode and the second of electrode;
a memory within the housing; and
first processing circuitry within the housing, the first processing circuitry configured to collect parameter data of the subject based on the sensed physiological signals; and
one or more computing devices in communication with the insertable cardiac monitor, the one or more computing devices comprising second processing circuitry configured to determine a mental state of the subject based on at least one of the sensed physiological signals or the parameter data.

16. A method for operating a system comprising one or more implantable monitoring devices to determine a mental state of a subject, wherein at least one implantable monitoring device of the one or more implantable monitoring devices comprises a housing configured for subcutaneous implantation in the subject and a plurality of electrodes positioned on the housing, wherein the at least one implantable monitoring device is configured to continuously sense at least one physiological signal of the plurality of physiological signals via the plurality of electrodes, the method comprising:
continuously sensing, by the one or more implantable monitoring devices, a plurality of physiological signals of the subject;
collecting, by the one or more implantable monitoring devices, parameter data of the subject based on the sensed physiological signals; and
determining, by processing circuitry, a mental state of the subject based on at least one of the sensed physiological signals or the parameter data, wherein the processing circuitry comprises processing circuitry of one or more of:
the at least one implantable monitoring device; one or more computing devices configured to wirelessly communicate with the one or more implantable monitoring devices; or
a cloud computing system configured to communicate with at least one of the one or more implantable monitoring devices or the one or more computing devices.

17. The method of claim 16, wherein the housing of the at least one implantable monitoring device is configured for subcutaneous implantation on a head or neck of the subject, and continuously sensing a plurality of physiological signals comprises continuously sensing an electroencephalogram (EEG) of the subject via the plurality of electrodes.

18. The method of claim 16, wherein the at least one implantable monitoring device comprises a first implantable monitoring device comprising a first housing and a first plurality of electrodes, wherein the one or more implantable monitoring devices comprise a second implantable monitoring device comprising a second housing configured for subcutaneous implantation on a head or neck of the subject and a second plurality of electrodes on the housing, wherein continuously sensing a plurality of physiological signals comprises continuously sensing an electroencephalogram (EEG) of the subject via the second plurality of electrodes.

19. The method of claim 17, wherein determining the mental state comprises determining the mental state of the subject based on a morphology of the EEG.

20. The method of claim 17, wherein determining the mental state comprises determining the mental state of the subject based on a respective energy level in one or more frequency bands or sensing locations of the EEG.
